(19) United States
(12) Patent Application Publication
(10) Pub. No.: US 2024/0387024 A1
(43) Pub. Date: Nov. 21, 2024

(54) SYSTEMS AND METHODS FOR DENTAL APPLIANCE AND DENTAL LAB QUALITY ASSESSMENT

(71) Applicant: VOYAGER DENTAL, INC., Eden Prairie, MN (US)

(72) Inventors: Stephen B. Siegfried FLOE, Chanhassen, MN (US); Michael C. MARSHALL, Edina, MN (US); John Michael MADDEN, Minneapolis, MN (US)

(21) Appl. No.: 18/584,643

(22) Filed: Feb. 22, 2024

Related U.S. Application Data
(60) Provisional application No. 63/486,442, filed on Feb. 22, 2023.

(51) Int. Cl. G16H 40/20; A61C 13/00

(52) U.S. Cl. CPC G16H 40/20; A61C 13/00

(57) ABSTRACT

Disclosed are techniques for determining a lab quality score. A dental lab computer system can generate instructions that cause a manufacturing machine to manufacture a dental appliance for a patient in a dental lab, and generate dental appliance data based on user input indicating information about manufacturing the dental appliance. A dental office computer system can generate dental appliance procedure data based on a dentist performing a procedure to install the dental appliance. A remote computer system in communication with the dental lab computer system and the dental office computer system can: receive the dental appliance data and dental appliance procedure data from the dental lab computer system and the dental office computer system, respectively, aggregate the data, determine a lab quality score for the dental lab, identify, based on the lab quality score, a quality issue for the dental lab, and generate recommendations to improve the lab quality score.

### CROSS-REFERENCE TO RELATED APPLICATIONS

This application is claims the priority benefit of U.S. Provisional Patent Application No. 63/486,442, filed Feb. 22, 2023, which is incorporated herein by reference in its entirety.

### TECHNICAL FIELD

This document generally describes devices, systems, and methods related to computer-based assessment and scoring of dental appliances and dental labs manufacturing dental appliances, such as dentures, dental implants, and other dental appliances.

### BACKGROUND

Dental appliances can be designed by healthcare providers like orthodontists and dentists and inserted into patients' mouths in dental appliance procedures. Such dental appliances can include but are not limited to teeth aligners, retainers, dentures, crowns, bridges, wires, and other types of structures that can be inserted in the patients' mouths. A dental appliance can be custom designed to fit a particular patient's mouth and teeth setup. The dental appliance can be designed using computer-automated software. Once the dental appliance is designed, files and/or instructions for manufacturing or fabricating the dental appliance can be sent to a dental lab. The dental lab can use the files and/or instructions to manufacture the dental appliance. Once the dental appliance is manufactured, the dental lab can send the dental appliance to a dentist or other healthcare provider who inserts the dental appliance into the particular patient's mouth.

The dental lab can use various types of equipment to manufacture different types of dental appliances, including but not limited to dental appliance presses, hydraulic presses, dental appliance curing units, boilout units, dental appliance compresses, flasks and presses, pressure pots, etc. The dental lab can also include fabrication machines such as 3D printers, which may also be used to manufacture custom dental appliances for patients. Manufacturing the dental appliances can include a combination of automated processes performed by devices, computers, robots, etc. as well as manual processes performed by a lab technician. For example, a 3D printer can be used to print a dental appliance according to the files and/or instructions for manufacturing the dental appliance. The lab technician can then manually apply paints to the dental appliance once printed to achieve desired coloration of the dental appliance.

### SUMMARY

The document generally describes technology for assessing and scoring dental appliances and dental labs that manufacture the dental appliances. More particularly, described are computer-automated techniques for determining dental lab quality scores for a plurality of dental labs, ranking the dental labs based on their quality scores, and providing a feedback mechanism for improving processes at the labs in manufacturing the dental appliances. A lab quality score for a particular lab can be determined based on a variety of weighted factors, including but not limited to accuracy in shaping and sculpting dental appliances, coloring the dental appliances, manual manipulation (e.g., by a lab technician) of the dental appliances, etc. The lab quality score can also be determined based on weighing in factors such as dentist records that indicate how many corrections the dentist made when fitting a dental appliance manufactured by the lab in a patient's mouth, whether the dentist sent the dental appliance back to the lab for adjustment, the patient's chair time during insertion of the dental appliance, whether the patient returned to the dentist for adjustments of the dental appliance, etc. Furthermore, metrics such as difficulty in manufacturing a particular type of dental appliance and/or complexity of the dental appliance can be used to score the particular lab's quality and rank the particular lab amongst the plurality of dental labs.

The determined lab quality scores can be used in a variety of applications. For example, the lab quality scores can be used to rank and assess dental labs across states and/or countries. The lab quality scores can be used to determine (e.g., by a computer system) one or more areas of improvement at a particular dental lab. Fixing the one or more areas of improvement can cause an increase in a lab quality score for the particular dental lab, thereby increasing the particular dental lab's overall ranking amongst the plurality of dental labs. Consequently, the disclosed technology can provide an automated feedback mechanism for highlighting issues in different dental labs, improving processes in those labs to resolve such issues, and thereby providing higher quality dental appliances to experiences for dentists, other healthcare providers, and patients. The lab quality scores can also be used to determine reimbursement rates for purposes of insurance coverage and policies. The lab quality scores can also be used to assess dentists and determine quality scores for the respective dentists. The disclosed technology can also be used to highlight issues in the respective dentists practices, based on their quality scores, and provide recommendations for improving their practices.

One or more embodiments described herein can include a system for determining a lab quality score, the system including: a dental lab computer system that can be configured to: generate instructions that, when executed, cause at least one manufacturing machine to manufacture a dental appliance for a patient in a dental lab, generate dental appliance data based at least in part on user input that is received from a lab technician indicating information about manufacturing the dental appliance for the patient, a dental office computer system that can be configured to generate dental appliance procedure data based on a dentist performing a procedure to install the dental appliance in the patient's mouth, and a remote computer system in communication with the dental lab computer system and the dental office computer system, the remote computer system being configured to: receive the dental appliance data and the dental appliance procedure data from the dental lab computer system and the dental office computer system, respectively, aggregate the received data, determine a lab quality score for the dental lab based at least in part on the aggregated data, identify, based on the lab quality score, at least one quality issue for the dental lab, generate at least one recommendation to improve the lab quality score of the dental lab based on the identified at least one quality issue, and return the lab quality score, the identified at least one quality issue, and the at least one recommendation for presentation in a graphical user interface (GUI) display of a computing device of a relevant user/

In some implementations, the embodiments described herein can optionally include one or more of the following features. For example, determining the lab quality score for the dental lab can include: generating a set of sub-scores and aggregating the sub-scores to determine the lab quality score. Generating the set of sub-scores can include: generating a sub-score that exceeds a threshold sub-score value based on an accuracy in shaping or sculpting the dental appliance at the dental lab satisfying one or more manufacturing criteria. Generating the set of sub-scores can include: generating a sub-score that exceeds a threshold sub-score value based on an accuracy of coloring the dental appliance at the dental lab satisfying one or more color criteria. Generating the set of sub-scores can include: generating a sub-score that exceeds a threshold sub-score value based on manufacturing practices of the dental lab satisfying one or more manufacturing criteria. Generating the set of sub-scores can include: generating a sub-score that exceeds a threshold sub-score value based on a quantity of corrections made to the dental appliance by the dentist during the procedure to install the dental appliance in the patient's mouth being less than a threshold quantity of corrections.

As another example, generating the set of sub-scores can include: generating a sub-score that exceeds a threshold sub-score value based on a type of corrections made to the dental appliance by the dentist during the procedure to install the dental appliance in the patient's mouth satisfying one or more correction-type criteria. Generating the set of sub-scores can include: generating a sub-score that exceeds a threshold sub-score value based on a total chair time of the patient during the procedure to install the dental appliance in the patient's mouth being less than a threshold amount of time. Aggregating the sub-scores to determine the lab quality score can include: weighting one or more of the sub-scores based on a difficulty assessment for manufacturing the dental appliance, the lab quality score exceeding a threshold score value when manufacturing the dental appliance has a difficulty level above a threshold difficulty level. Aggregating the sub-scores to determine the lab quality score can include: weighting one or more of the sub-scores based on a complexity of the dental appliance, the lab quality score exceeding a threshold score value when the complexity of the dental appliance is greater than a threshold level of complexity.

In some implementations, the remote computer system can also determine a dentist quality score for the dentist based on at least one of the aggregated data or the lab quality score. Determining the dentist quality score for the dentist can include: assigning the dentist quality score a value that exceeds a threshold score value based on a total patient chair time associated with the dentist being less than a threshold chair time value. Determining the dentist quality score for the dentist can include: assigning the dentist quality score a value that exceeds a threshold score value based on the lab quality score exceeding a threshold lab quality score value. The remote computer system can also retrieve, from a data store, lab quality scores for a group of dental labs that includes the dental lab, rank the group of dental labs that includes the dental lab based on the respective lab quality scores into a list of ranked dental labs, and return the list of ranked dental labs. The relevant user can be another patient. The relevant user can be a third party insurance provider. The relevant user can be another dentist or another dental lab.

One or more embodiments described herein can include a method for determining a lab quality score, the method including: receiving, by a remote computer system, dental appliance data and dental appliance procedure data from a dental lab computer system and a dental office computer system, respectively, the dental appliance data being based at least in part on user input that can be received from a lab technician at the dental lab computer system indicating information about manufacturing a dental appliance for a patient, the dental appliance procedure data being based on a dentist performing a procedure to install the dental appliance in the patient's mouth, aggregating, by the remote computer system, the received data, determining, by the remote computer system, a lab quality score for a dental lab that is associated with the dental appliance data based at least in part on the aggregated data, identifying, by the remote computer system and based on the lab quality score, at least one quality issue for the dental lab, generating, by the remote computer system, at least one recommendation to improve the lab quality score of the dental lab based on the identified at least one quality issue, and returning, by the remote computer system, the lab quality score, the identified at least one quality issue, and the at least one recommendation for presentation in a graphical user interface (GUI) display of a computing device of a relevant user.

The method can optionally include one or more of the abovementioned features.

The devices, system, and techniques described herein may provide one or more of the following advantages. For example, the disclosed technology provides for more efficiently processing various information and data about manufacturing dental appliances, practices of dental labs, and dentist procedure records to efficiently and accurately assess and score a particular dental lab based on quality. The disclosed technology can distill a set of complex information and data to a single metric, such as a single quality score or multiple sub-scores that can then be used to infer overall quality of the particular dental lab relative to other dental labs.

As another example, the disclosed technology provides a standardized process for consistently assessing and scoring quality of dental labs practices across states and countries. The disclosed technology standardizes an inherently subjective process for assessing quality of dental labs. The disclosed technology also helps practitioners in dentistry better and more accurately determine which labs to partner with for manufacturing patients' dental appliances.

The details of one or more implementations are set forth in the accompanying drawings and the description below. Other features and advantages will be apparent from the description and drawings, and from the claims.

### BRIEF DESCRIPTION OF THE DRAWINGS

FIG. 1 is a conceptual diagram for assessing a dental lab and determining a lab quality score for the dental lab.

FIGS. 2A-B is a flowchart of a process for determining a dental lab quality score.

FIG. 3 is a flowchart of a process for ranking a plurality of dental labs based on their respective quality scores and identifying one or more issues that may be addressed to improve quality scores and overall ranking for one or more of the plurality of dental labs.

FIG. 4 is a flowchart of a process for determining a dentist quality score.

FIGS. 5A-B are example graphical user interface (GUI) displays for outputting information about dental labs and/or dentists according to their respective quality scores.

FIG. 6 is a system diagram of one or more components that can be used to perform the disclosed techniques.

FIG. 7 is a schematic diagram that shows an example of a computing device and a mobile computing device.

Like reference symbols in the various drawings indicate like elements.

### DETAILED DESCRIPTION OF ILLUSTRATIVE EMBODIMENTS

This document generally relates to computer-automated techniques for determining lab quality scores for a plurality of dental labs, ranking the dental labs based on their quality scores, and providing a feedback mechanism for improving processes at the labs in manufacturing the dental appliances. A lab quality score for a particular lab can be determined based on a variety of factors and sources of information or data, including manufacturing records from the lab and dentist records from dentists who use the dental appliances manufactured by the lab. Lab quality scores can also be used to assess dentists and determine respective dentist quality scores.

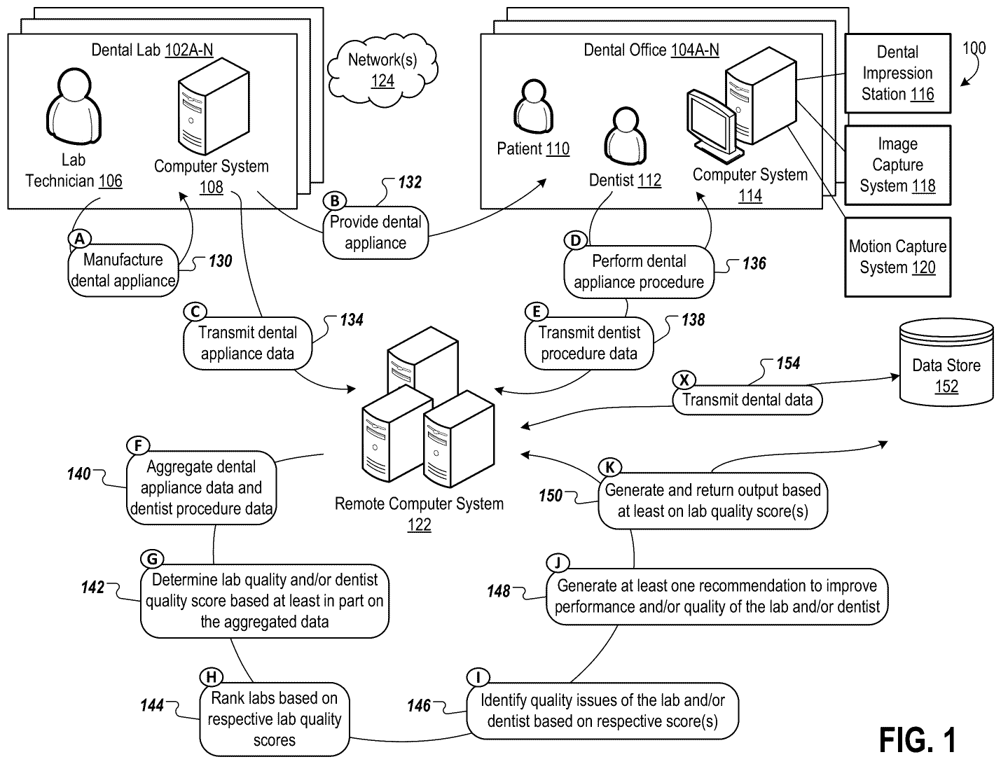

Referring to the figures, FIG. 1 is a conceptual diagram of a system 100 for assessing a dental lab and determining a lab quality score for the dental lab. In the system 100, a computer system 108, a computer system 114, a remote computer system 122, and/or a data store 152 may communicate (e.g., wired and/or wireless) via network(s) 124. The computer systems 108, 114, and 122 can be any type of computing devices, networks of computing devices and/or systems, and/or cloud-based systems described herein.

The computer system 108 can be configured to collect, determine, generate, and/or receive data about processes performed by a lab technician 106 in respective dental labs 102A-N. Such processes can include manufacturing dental appliances. Each dental lab 102A-N can have a respective computer system 108. In some implementations, the computer system 108 can be associated with multiple dental labs 102A-N and can be remote from one or more of the dental labs 102A-N.

The computer system 114 can be configured to collect, determine, generate, and/or capture data about a patient 110 in a respective dental office 104A-N. Each dental office 104A-N can have a respective computer system 114. In some implementations, the computer system 114 can be associated with multiple dental offices 104A-N and can be remote from one or more of the dental labs 104A-N.

The remote computer system 122 can be configured to receive data from the computer systems 108 and 114 and use the data to assess and score the dental labs 102A-N based on one or more quality metrics and/or factors. The remote computer system 122 can also use the data to assess and score dentists 112 in the dental offices 104A-N. In some implementations, one or more of the computer system 108, the computer system 114, and/or the remote computer system 122 can be part of a same computer system or cloud-based system.

The computer system 114 can communicate via the network(s) 124 with a dental impression station 116, an image capture system 118, and a motion capture system 120. Sometimes, the computer system 114 can be integrated into or otherwise part of at least one of the dental impression station 116, the image capture system 118, and the motion capture system 120. The computer system 114 can generate instructions that cause the dental impression station 116, the image capture system 118, and/or the motion capture system 120 to capture data of the patient 110 in the respective dental office 104A-N.

The example dental impression station 116 is configured to generate a dental impression of dentition of the patient 110. The dental impression can be a geometric representation of the dentition of the patient 110, which may include teeth (if any) and edentulous (gum) tissue, or gingiva as described herein. In some implementations, the dental impression can be a physical impression captured using an impression material, such as sodium alginate, polyvinylsiloxane or another impression material

The image capture system 118 can be configured to capture image data of the patient 110's mouth. The image data may include one or more static images or videos of the patient 110. The static images or frames with the image data may be associated with motion data. For example, a specific image from the image data may be associated with a specific frame of the motion data, indicating that the specific image was captured while the patient 110's jaw was in a position indicated by the specific frame of the motion data. In some implementations, the image capture system 118 includes a three-dimensional (3D) camera and the image data may include one or more 3D images. Examples of 3D cameras include stereo cameras (e.g., using two or more separate image sensors that are offset from one another). The 3D camera may also include a projector such as a light projector or laser projector that operates to project a pattern on the patient 110's face. For example, the projector may be offset relative to the camera or cameras so that the images captured by the camera include distortions of the projected pattern caused by the patient 110's face. Based on these distortions, the three-dimensional structure of portions of the patient 110's face can be approximated. Various implementations project various patterns such as one or more stripes or fringes (e.g., sinusoidally changing intensity values). In some implementations, the 3D image is captured in relation to the motion capture system 120 or a portion thereof so that the 3D images can be related to the same coordinate system as the motion data.

The motion capture system 120 can be configured to capture a representation of movement of dental arches relative to each other in the patient 110's mouth. In some implementations, the motion capture system 120 generates the motion data. The dental impression can also be used to generate a patient-specific dentition coupling device for capturing patient motion using the motion capture system 120. Some implementations described herein may use other types of motion capture systems to generate motion data of the patient 110's mouth.

The data store 152 can be any type of data storage system, database, data repository, and/or memory. The data store 152 can be configured to store various types of dental data, including but not limited to any of the data and/or information that is collected and/or generated by the components 108, 114, 116, 118, 120, and/or 122. The data store 152 can maintain information including but not limited to dental appliance manufacturing instructions, dental appliance models, patient dental models, patient records, lab records, lab technician information, dentist records, dentist information, dental impression data, image data of the patient's teeth and/or dental appliances, motion data for the patient, patient procedure data, or any other types of information described herein that may be used with the disclosed techniques. The data store 152 can maintain data for a variety of the dental labs 102A-N and/or the dental offices 104A-N, regardless of their geographic location. Sometimes, the data store 152 can be part of the remote computer system 122. The data store 152 can also be part of one or more of the computer systems 108, 114, and/or 122 described herein. In some implementations, each of the computer systems 108, 114, and/or 122 can communicate with a different data store, rather than one centralized data store 152.

Still referring to the system 100 in FIG. 1, the lab technician 106 can manufacture a dental appliance at a dental lab 102 (block A, 130). The dental appliance can include but is not limited to dentures, crowns, bridges, aligners, wires, or other types of appliances that can be manufactured for a patient to resolve a dental issue of the patient. The lab technician 106 can use instructions to manufacture the dental appliance. The instructions can be received by the computer system 108 from the remote computer system 122 or the computer system 114 of a dental office 104 that requests the dental appliance for the patient 110. The instructions can be generated by the remote computer system 122 and/or the computer system 114 based on patient data that is captured by the dental impression station 116, the image capture system 118, and the motion capture system 120 and used to generate a 3D model of the dental appliance for the patient 110.

The instructions used to manufacture the dental appliance can include instructions for controlling one or more automated or semi-automated machines for manufacturing dental appliances in the dental lab 102. For example, the instructions can cause a 3D printer or other fabrication machine to manufacture the dental appliances according to particular measurements in the 3D model of the dental appliance for the patient 110. The instructions can additionally or alternatively include steps for the lab technician 106 to perform in manually manufacturing, molding, coloring, etc. the dental appliance.

Once the dental appliance is manufactured at the dental lab 102, the dental appliance can be provided to the dental office 104 (block B, 132).

The computer system 108 of the dental lab 102 can also transmit dental appliance data to the remote computer system 122 (block C, 134). The dental appliance data can include information about the process to manufacture the dental appliance. For example, the dental appliance data can indicate how long it took to manufacture the dental appliance, how many corrections and/or actions the lab technician 106 took while manufacturing the dental appliance, whether multiple versions of the dental appliance had to be manufactured to comply with the expected measurements for the dental appliance, etc. The dental appliance data can include the 3D model for manufacturing the dental appliance, the instructions for manufacturing the dental appliance, expected measurements for the dental appliance, actual measurements of the dental appliance post-manufacturing, etc. Block C (134) can be performed while the dental appliance is being manufactured in block A (130) and/or before providing the dental appliance to the dental office 104 in block B (132).

Once the dental appliance is received at the dental office 104, the dentist 112 can perform a dental appliance procedure in block D (136). The dental appliance procedure can include inserting the dental appliance into the patient 110's mouth. The dental appliance procedure can also include performing, by the dentist 112, any modifications, corrections, and/or adjustments of the dental appliance. For example, the dentist 112 may modify the dental appliance if the dental appliance does not fit correctly into the patient 110's mouth. Sometimes, the dental appliance may not be accurately manufactured at the dental lab 102, and thus may be defective in some way. The defective dental appliance may still be provided to the dental office 104, but the dentist 112 may have to adjust the dental appliance to rectify one or more defects from the manufacturing process. In some implementations, the dentist 112 may even send the defective dental appliance back to the dental lab 102 if the dentist 112 is unable to adjust the dental appliance and/or the adjusted dental appliance still does not work well for the patient 110. As described herein, actions performed by the dentist 112 to modify and insert the dental appliance into the patient 110's mouth can be used as inputs by the remote computer system 122 to determine a quality score for the lab 102 that manufactured the dental appliance.

As part of or following the dental appliance procedure, the computer system 114 can transmit dentist procedure data to the remote computer system 122 (block E, 138). The dentist procedure data can include records that are generated/made by the dentist 112 before, during, and/or after the dental appliance procedure. For example, the dentist procedure data can indicate how long the patient 110 was sitting in the chair from start to finish of the dental appliance procedure, whether the dental appliance fit in the patient 110's mouth, whether the dentist 112 modified or adjusted the dental appliance before, during, or after inserting the dental appliance into the patient 110's mouth, what type of modifications or adjustments the dentist 112 made, how long it took to make the modifications or adjustments, whether the patient 110 returned after the procedure to get the dental appliance adjusted or readjusted, etc. One or more other information can also be included in the dental procedure data.

In some implementations, the dental appliance data and/or the dentist procedure data can be stored in the data store 152 and then later accessed/retrieved by the remote computer system 122 to perform the operations described below.

The remote computer system 122 can aggregate the dental appliance data and the dentist procedure data in block F (140). Aggregating the received data can include identifying associations or relationships between the data, such as identifying which lab technician in which of the dental labs 102A-N manufactured the dental appliance that is being inserted not the patient 110's mouth in one of the dental offices 104A-N. The remote computer system 122 can also aggregate the data using one or more machine learning models, algorithms, or other techniques to identify various types of relationships and associations amongst the large amounts of data that the remote computer system 122 receives in at least blocks C and E (134 and 138 respectively).

The remote computer system 122 can also receive dental data from the data store 152 at any time during the disclosed techniques (block X, 154). For example, the remote computer system 122 can receive patient data, such as a 3D dental model of the patient 110's mouth before and/or after the dentist 112 performs the dental appliance procedure in block D (136). The remote computer system 122 can aggregate the dental data received (or retrieved) from the data store 152 with the other received data in block F (140). Furthermore, any determinations made by the remote computer system 122 or other generated data can be transmitted to the data store 152 for storage at any time during the disclosed techniques (block X, 154).

The remote computer system 122 can determine a lab quality score for the dental lab 102 based on the aggregated data (block G, 142). The lab quality score can be generated using machine learning models, algorithms, or other techniques. The lab quality score can be a value or metric that quantifies an ability of the dental lab 102 to manufacture the dental appliance, a plurality of dental appliances, and/or a particular type of dental appliance based on their respective instructions for manufacturing. The lab quality score can be a numeric value on a scale, such as a scale of 0 to 100, where a score of 100 indicates highest quality and a score of 0 indicates lowest quality. The lab quality score can be an aggregate of multiple sub-scores, in some implementations. For example, the lab quality score can be based on assessments of shaping, sculpting, coloring, and other practices performed manually by the lab technician 106 and/or automatically by manufacturing equipment in the dental lab 102 when manufacturing the dental appliance or a plurality of dental appliances.

The lab quality score can be based on the data that was aggregated in block F (140). For example, the lab quality score can be at least partially based on information recorded by the dentist 112 during the dental appliance procedure in the dentist procedure data. The lab quality score can be based at least in part on how many corrections the dentist 112 had to make in order to insert the dental appliance into the patient 110's mouth. The lab quality score can be based at least in part on whether and how much the dentist 112 had to mold, shape, or otherwise modify the dental appliance to fit into the patient 110's mouth. The lab quality score can be based at least in part on how long the patient 110 is in the chair from start to end of the dental appliance procedure. The lab quality score can be based at least in part on whether the dentist 112 sends the dental appliance back to the dental lab 102 because the dental appliance is rejected from the patient 110's mouth or otherwise does not fit correctly in the patient 110's mouth. The more corrections or modifications that the dentist 112 has to make with respect to the dental appliance, the lower the quality score determined for the dental lab 102. Conversely, the less time the patient 110 spends in the chair, the fewer modifications to the dental appliance, and/or the patient 110 not having to return to the dentist 112 to fix the dental appliance post-procedure, can contribute to the remote computer system 122 determined a higher lab quality score for the dental lab 102.

In some implementations, the lab quality score can be determined and/or weighted based on a difficulty assessment of the particular dental appliance and/or a process for manufacturing the dental appliance. The difficulty assessment can be performed based on assessing a population of data, records, or other information provided by the computer systems of multiple different dental labs and/or dental offices. For example, some types of dental appliances (e.g., crowns, bridges, wires, aligners, dentures, etc.) can be more or less difficult to manufacture than other dental appliances. If the dental lab 102, for example, manufactures a complex type of crown, then the dental lab's quality score can be weighted more than a quality score of another dental lab that manufactures simple wires, among other factors described herein for determining and weighting the lab quality score. As yet another example, a high number of corrections performed by the dentist 112 for a simple dental appliance can result in a lower quality score for the respective dental lab.

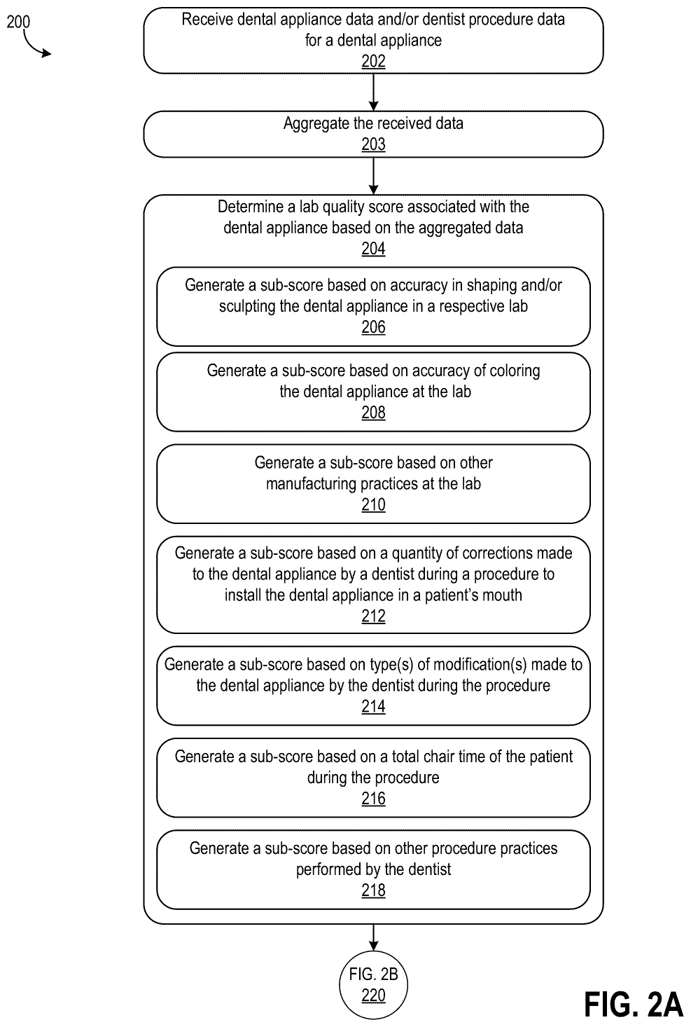

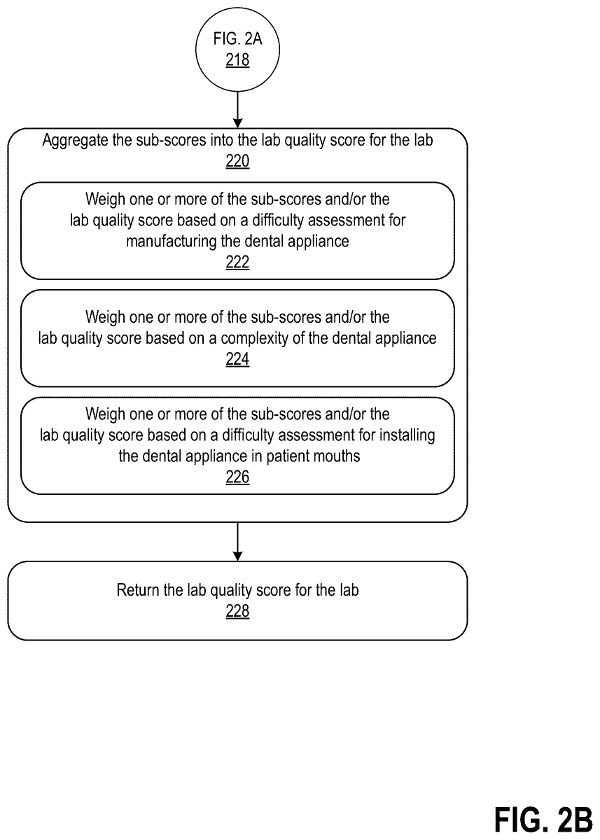

The lab quality score can additionally or alternatively be a Boolean value or string value that indicates a level of quality of the dental lab's dental appliance manufacturing techniques/processes. Refer to FIGS. 2A-B for further discussion about generating the lab quality score.

Additionally or alternatively, the remote computer system 122 can determine a dentist quality score for the dentist 112 based at least in part on the aggregated data (block G, 142). Sometimes, for example, the dentist quality score can be generated based at least in part on the lab quality score that was also determined. The remote computer system 122 can generate quality scores for dentists in a plurality of dental offices 104A-N. The remote computer system 122 can use the dentist quality scores to identify outlier dentists. Various factors can be used, aggregated, and/or weighted by the remote computer system 122 to determine the quality score for the dentist 112. For example, the dentist quality score can be determined based at least in part on a total chair time of one or more patients who see the dentist 112 for dental appliance procedures. The more chair time (per patient and/or in the aggregate) can be used to generate a lower quality score for the dentist 112 than if the dentist 112 is more efficient in quickly and accurately completing the dental appliance procedures. The dentist quality score can be determined based at least in part on whether one or more patients return to the dentist 112 for further adjustments. For example, the more patients who come in for adjustments post-procedure can be used to determine a lower quality score for the dentist 112 than if fewer patients return to the dentist 112 for adjustments. The dentist quality score can be determined based at least in part on a complexity of the procedure to insert the dental appliance and/or a complexity of the dental appliance. The more complex the dental appliance, for example, the higher the quality score for the dentist 112. Various other factors may also be used and weighted to determine the dentist quality score.

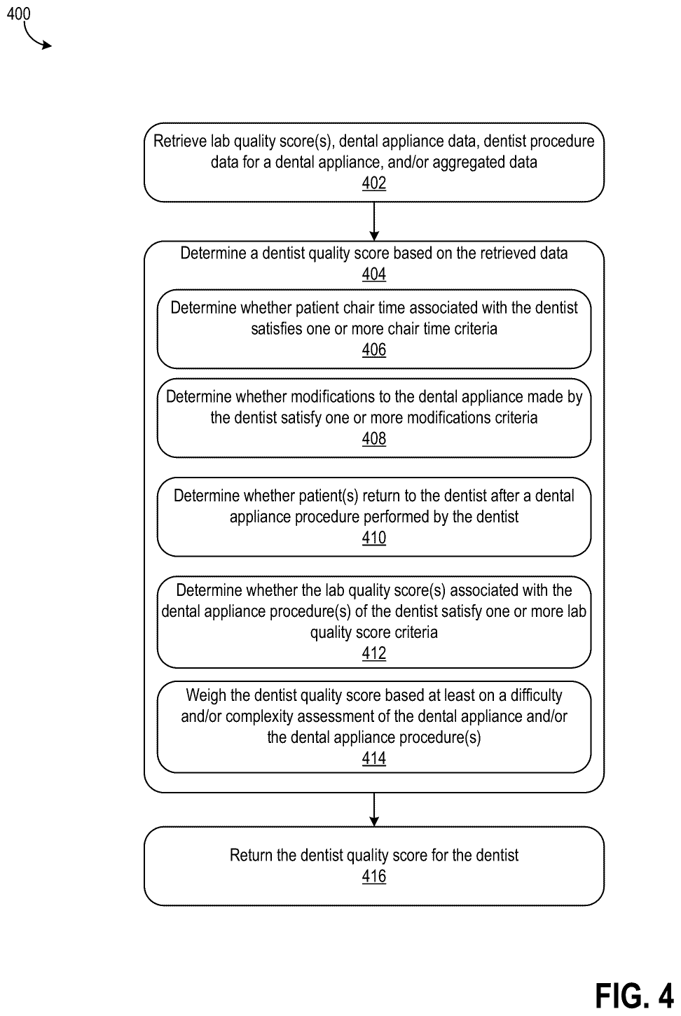

The dentist quality score can be a numeric value on a scale, such as a scale of 0 to 100, where a score of 100 indicates highest quality and a score of 0 indicates lowest quality. The dentist quality score can be an aggregate of multiple sub-scores, in some implementations. The dentist quality score can additionally or alternatively be a Boolean value or string value that indicates a level of quality of the dentist in performing one or more types of dental appliance procedures. Refer to FIG. 4 for further discussion about scoring the dentist 112.

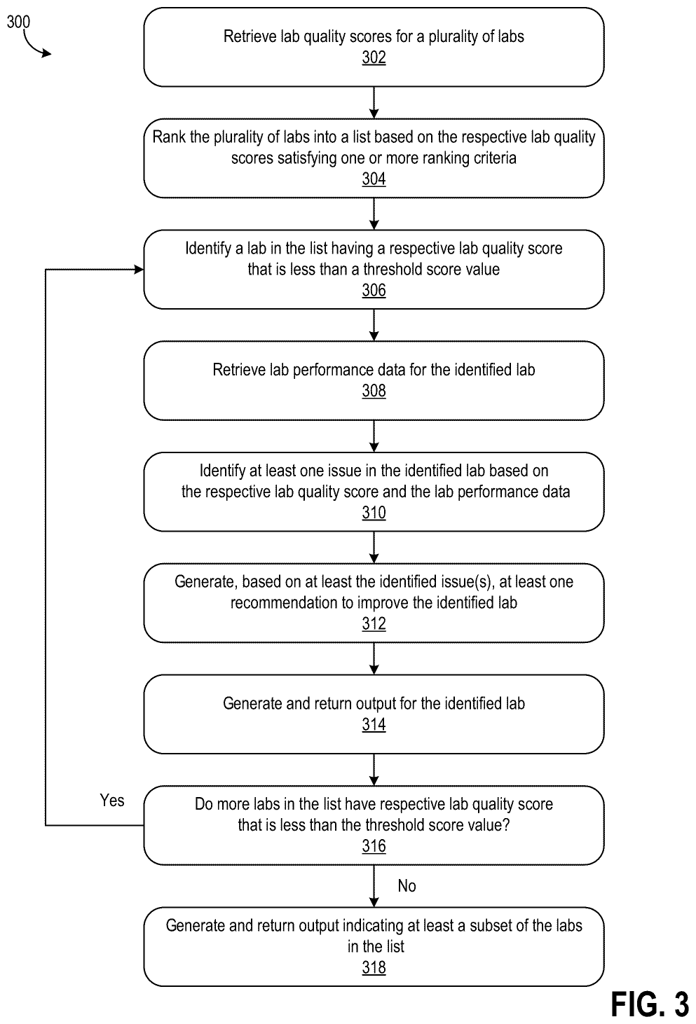

In block H, the remote computer system 122 can rank the dental labs 102A-N based on their respective lab quality scores (block H, 144). The dental labs 102A-N in a particular region can be ranked amongst each other. The dental labs 102A-N across a particular country or across the world can be ranked amongst each other. The dental labs 102A-N that manufacture a particular type of dental appliance can additionally or alternatively be ranked amongst each other. One or more other factors can be used to determine which dental labs to rank amongst each other based on their respective lab quality scores. The ranked dental labs can then be used by relevant users to determine which labs have highest quality output and thus should be used for manufacturing certain types of dental appliances. Refer to FIG. 3 for further discussion.

The remote computer system 122 can identify at least one quality issue of the dental lab 102 and/or the dentist 112 based on the respective quality scores (block I, 146). The remote computer system 122 can use machine learning techniques and/or models to analyze the dental appliance data, the dentist procedure data, and/or the determined quality scores and identify potential areas for improvement in the dental lab 102 and/or the dentist 112's procedures. The remote computer system 122 can determine, for example, particular techniques performed by the lab technician 106 that can be performed more precisely and/or more efficiently. As another example, the remote computer system 122 can determine one or more steps that the dentist 112 can perform before requesting the dental appliance to be manufactured at the dental lab 102 in an effort to reduce an amount of manual manipulation to be performed by the lab technician 106 while manufacturing the dental appliance and/or to reduce an amount of modifications performed by the dentist 112 at the time of the dental appliance procedure. Refer to FIG. 3 for further discussion.

The remote computer system 122 can also generate at least one recommendation to improve performance and/or quality of the dental lab 102 and/or the dentist 112 (block J, 148). The recommendation(s) can be generated using machine learning models, techniques, rules, and/or one or more recommendation criteria. The remote computer system 122 can identify a factor that contributed the most to the lab quality score. The factor can be, for example, manually coloring dentures during the denture manufacturing process. The remote computer system 122 can determine that the dentures should be colored automatically by a machine in the dental lab 102, rather than being manually colored by the lab technician 106. Therefore, the remote computer system 122 can generate a recommendation indicating that one or more types of dentures that are manufactured at the dental lab 102 should be automatically colored by a machine rather than the lab technician 106. In some implementations, as part of generating the recommendation(s), the remote computer system 122 may also determine how much the lab quality score (or the dentist quality score) will improve/increase based on the recommendation being implemented. This information can beneficially be used by the dental lab 102 and/or the dental office 104 in determining whether to implement the recommendation.

As another example, in block J (148), the remote computer system 122 can determine and/or set insurance reimbursement rates based on the quality score for the dental lab 102 and/or the dentist 112. If the dental lab 102 does a high quality job in manufacturing the dental appliance and the dentist 112 uses less time to insert the dental appliance into the patient 110's mouth as a result of the high quality work of the dental lab 102, then insurance costs can be reduced, which can incentivize other dentists, dental offices, and/or patients to work with the dental lab 102. Refer to FIG. 3 for further discussion about block J (148).

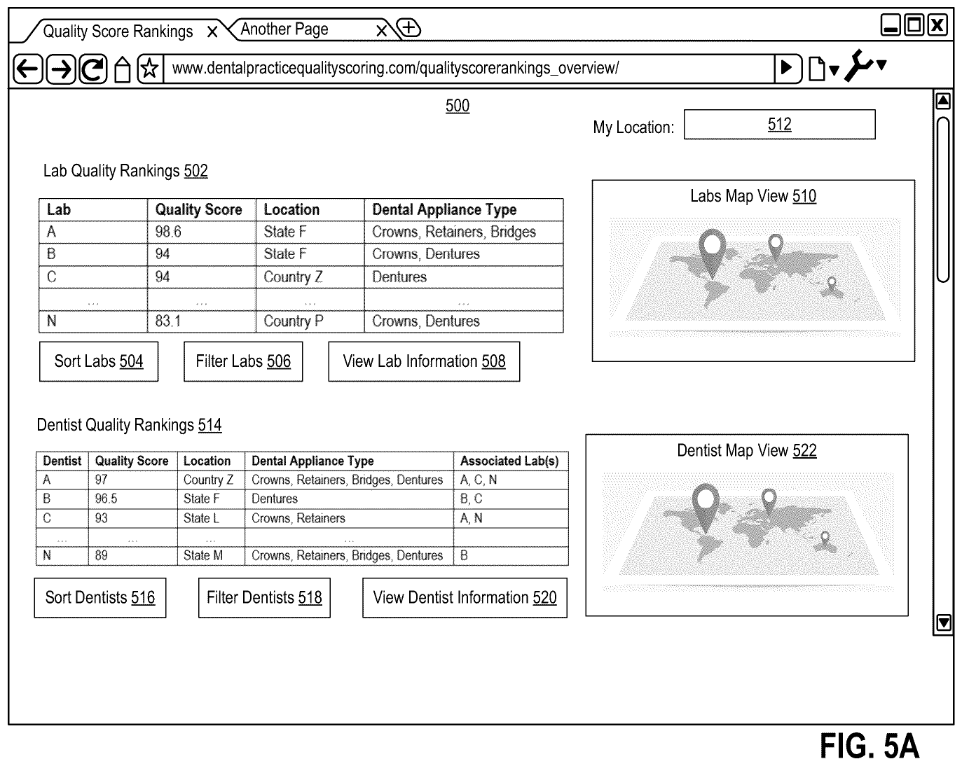

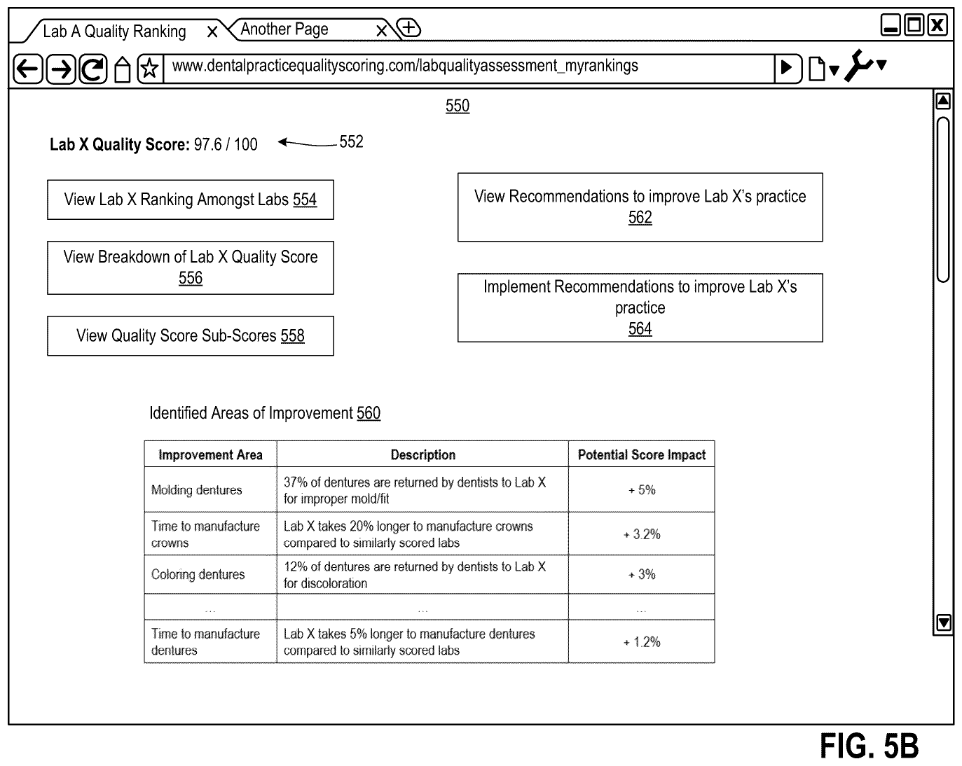

Accordingly, the remote computer system 122 can generate and return output based at least on the lab quality score for the dental lab 102 (and/or lab quality scores for a group of the dental labs 102A-N) (block K, 150). The output can be returned to the computer system 108 and presented in a graphical user interface (GUI) display at the computer system 108 for the lab technician 106's review. The output can be returned to the computer system 114 and presented in a GUI display at the computer system 114 for the dentist 112's review. In some implementations, the output can be returned to computing systems and/or devices of other relevant users, including but not limited to lab technicians in one or more of the other dental labs 102A-N and/or dentists in one or more of the other dental offices 104A-N. In yet some implementations, the output can be returned to computing systems and/or devices of patients and/or other users who would like to pick a dentist and/or lab to work with in order to get their respective dental appliances manufactured and inserted. The output may also be transmitted to the data store 152 for storage in block K (150). Refer to FIGS. 5A-B for illustrative examples of the returned output.

The blocks F-K (140-150) can be performed at times that are different than when blocks A-E (130-138) are performed. For example, blocks A-C(130-134) can be performed at a first time, blocks D-E (136-138) can be performed at a second time, and blocks F-K (140-150) can be performed at a third time. The second time can occur some time after the first time. For example, it can take several days for the dental appliance to be delivered to the dental office 104 (block B, 132), and once the dental office 104 has the dental appliance, the dental appliance procedure may not occur for another several days, weeks, etc. The third time can occur some time after the third time. For example, the third time can be immediately after the second time. The third time can also be days, weeks, etc. after the second time and/or the first time. The remote computer system 122 may, for example, perform the blocks F-K (140-150) to score a group of the labs 102A-N at predetermined time intervals. The predetermined time intervals can include but are not limited to once a week, once a month, once a quarter, etc. In some implementations, the remote computer system 122 can perform the blocks F-K (140-150) to score the lab 102 once a threshold amount of dental appliance procedures are performed in dental offices 104A-N with dental appliances that are manufactured by the dental lab 102. As another example, the remote computer system 122 can perform the blocks F-K (140-150) to score the lab 102 once a threshold amount of dental appliance procedures are performed in dental offices 104A-N with a particular type of dental appliance that is manufactured by the dental lab 102. One or more other variations in performing the blocks A-K (130-150) are also possible.

FIGS. 2A-B is a flowchart of a process 200 for determining a dental lab quality score. The process 200 can be performed by the remote computer system 122 described herein. In some implementations, the process 200 can be performed by other computing systems, devices, computers, networks, cloud-based systems, and/or cloud-based services. For illustrative purposes, the process 200 is described from the perspective of a computer system.

Referring to the process 200 in both FIGS. 2A-B, the computer system can receive dental appliance data and/or dentist procedure data for a dental appliance in block 202. The data can be received from computer systems of one or more dental labs and/or one or more dental offices, as described in reference to FIG. 1. The data can additionally or alternatively be retrieved from a data store. The data can be received as it is generated at any of the computer systems described herein (e.g., dentist procedure data that is recorded by a dentist during a dental procedure for a particular patient). The data can also be retrieved at predetermined time intervals or at another time. For example, the computer system can retrieve the data from any of the computer systems and/or data store described herein at a time when the computer system is performing operations to score a particular dental lab and/or a particular dentist. The time can be, for example, every 24 hours, every day, once a week, once a month, or any other predetermined time.

Sometimes, for example, the particular dental lab can be scored every time that the dental lab manufactures a dental appliance, a particular type of dental appliance, and/or a threshold quantity of dental appliances. At such time, the computer system can poll the computer system at the particular dental lab for any relevant data that was generated before and/or as part of manufacturing the dental appliance. At such time, the computer system can retrieve the relevant data from the data store described herein. Similarly, the particular dental office can be scored every time that the dentist at the office performs a particular dental appliance procedure, a threshold quantity of procedures, etc. At such time, the computer system can poll the computer system at the particular dental office for any relevant data that was generated before, during, and/or after the procedure. At such time, the computer system can retrieve the relevant data from the data store described herein.

The computer system can aggregate the received data in block 203. Refer to block F (140) in FIG. 1 for further discussion.

In block 204, the computer system determines a lab quality score associated with the dental appliance and based on the received data. The computer system can generate the score based on assessing a variety of factors associated with the particular dental lab, one or more types of dental appliances that are manufactured by the lab, and/or one or more procedures that were performed for patients receiving dental appliances that are generated by the dental lab.

Sometimes, the lab quality score can be generated based on a plurality of sub-scores. In other words, the computer system can analyze the received data and assign scores to different factors represented by the received data. The computer system can then use one or more weights to combine/aggregate the sub-scores into the lab quality score for the particular dental lab.

For example, the computer system can generate a sub-score based on accuracy in shaping and/or sculpting the dental appliance in a respective dental lab (block 206). The dental lab can be scored based on manufacturing a particular type of dental appliance. The dental lab can also be scored based on manufacturing more than one type of dental appliance. In some implementations, the dental lab's score can also vary based on a difficulty level associated with manufacturing a particular type of dental appliance. The computer system at the dental lab and/or one or more devices that are used in the lab to manufacture the dental appliance (e.g., a 3D printer, another type of fabrication machine, etc.) can generate data about the manufacturing process of the particular dental appliance. One or more lab technicians in the dental lab can also generate data about the manufacturing process. The data can indicate, for example, expected measurements, size, and/or shape of the particular dental appliance. The data can additionally indicate actual measurements, size, and/or shape of the particular dental appliance. Sometimes, data generated by the lab technician(s) can indicate how many manual modifications were made by the lab technician(s) in order to improve accuracy of the shape, sculpt, and/or design of the dental appliance once it's manufactured. Sometimes, the data can include a 3D model and/or printing/manufacturing instructions that are used in the dental lab to manufacture the dental appliance.

The computer system can receive any of the abovementioned data and compare the expected measurements, size, and/or shape to the actual measurements, size, and/or shape of the particular dental appliance. The computer system can apply one or more rules to determine the sub-score based on this comparison. For example, the computer system can generate a higher sub-score (e.g., a score value between 50 and 100) based on the actual values being closer to the expected values. The computer system can generate a lower sub-score (e.g., a score value between 0 and 50) based on the actual values deviating more from the expected values. The computer system can generate multiple sub-scores, where each sub-score corresponds to a different aspect of the manufacturing process. For example, the computer system can generate a sub-score for accuracy of the dental appliance's shape and another sub-score for accuracy of the sculpting of the dental appliance. As mentioned above, the computer system can generate a sub-scores for the dental lab for each type of dental appliance that is manufactured at the dental lab. The sub-score per dental appliance can be weighted differently based on a difficulty level of manufacturing the particular type of dental appliance.

Additionally or alternatively, the computer system can generate a sub-score based on an accuracy of coloring the dental appliance at the respective dental lab (block 208). The computer system can receive data indicated expected color(s) of the dental appliance. The data can be, for example, the 3D model of the dental appliance and/or instructions for manufacturing the dental appliance. The computer system can receive data from the dental lab indicating what colors were used. The computer system can receive data based on manual inspection of the dental appliance by the lab technician and/or the dentist. The computer system can receive image data of the dental appliance after it is manufactured. The computer system can apply one or more rules and/or machine learning algorithms to this data in order to perform a comparison of expected coloring to actual coloring of the dental appliance. The more the actual coloring deviates from the expected coloring, the lower sub-score value the computer system may generate. As described herein, the computer system can generate multiple sub-scores based on coloring. For example, the computer system can generate a sub-score for coloring of a particular dental appliance for a particular patient. As another example, the computer system can generate a sub-score for coloring of a particular type of dental appliance that is manufactured for various patients. As yet another example, the computer system can generate a sub-score for coloring of all dental appliances manufactured at the particular dental lab over a threshold period of time.

Additionally or alternatively, the computer system can generate a sub-score based on other manufacturing practices at the respective dental lab (block 210).

Additionally or alternatively, the computer system can generate a sub-score based on a quantity of corrections made to the dental appliance by a dentist during a procedure to install the dental appliance in a patient's mouth (block 212). The more corrections made by the dentist, the lower the sub-score and consequently the lower the quality score for the dental lab that manufactured the dental appliance. Data can be received from the computer system of one or more dental offices that perform procedures with the dental appliances that are manufactured by the dental lab. The data can include input from the dentist performing the procedure. The input can indicate how long it took the dentist to perform the procedure, what type of corrections/modifications the dentist had to make to the dental appliance before, during, and/or after the procedure, how many corrections/modifications the dentist made, whether the patient returned for an adjustment after the procedure, how long after the procedure the patient returned, a complexity of the adjustments made/performed by the dentist, etc. Moreover, the computer system can generate or adjust the sub-score based on a complexity of the dental appliance. For example, if the dental appliance is complex and the dentist had to make more than a threshold quantity of adjustments because the dental appliance did not fit properly for the particular patient, then the computer system can assign a lower sub-score value than if the dental appliance is complex but the dentist made less than the threshold quantity of adjustments. In other words, the dental lab can receive a higher quality score if it manufactures complex dental appliances that rarely are adjusted during installation procedures than if it manufactures (i) complex appliances that are adjusted many times during the installation procedures or (ii) less complex appliances that are adjusted many times during the installation procedures.

Additionally or alternatively, the computer system can generate a sub-score based on type(s) of modification(s) made to the dental appliance by the dentist during the procedure (block 214). As mentioned above regarding block 212, the more complex type(s) of modification(s) made during the procedure, the lower the sub-score and thus the lower the quality score for the dental lab that manufactured the dental appliance. The dentist can provide input at their computer system in the dental office indicating the type(s) of modification(s) they made during the procedure. In some implementations, the computer system can also assess data provided by the dentist after the procedure, such as modifications that the dentist made to the dental appliance when the patient has returned to the dental office for further adjustments.

Additionally or alternatively, the computer system can generate a sub-score based on a total chair time of the patient during the procedure (block 216). Data can be provided as input by the dentist. Data can be generated by the computer system at the dental office, such as when the patient checks in and checks out of the dental office for their procedure appointment. The longer the patient is in the office, or more particularly in the chair at the dental office, the lower the sub-score and thus the lower the quality score for the dental lab. The less time the patient spends in the chair getting the dental appliance fitted and installed in their mouth, the more accurate the dental appliance that was manufactured by the dental lab, the higher the sub-score, and the higher the overall quality score for the dental lab.

Additionally or alternatively, the computer system can generate a sub-score based on other procedure practices performed by the dentist (block 218).

Next, in block 220, the computer system aggregates any one or more of the sub-scores into the lab quality score for the respective dental lab. The computer system can sum one or more of the sub-scores. Additionally, the computer system may average one or more of the sub-scores. The computer system can apply one or more rules to determine how to aggregate one or more of the sub-scores and/or weight one or more of the sub-scores.

As part of aggregating the sub-scores, the computer system may weigh one or more of the sub-scores and/or the lab quality score based on a difficulty assessment for manufacturing the dental appliance (block 222). In some implementations, the more difficult the manufacturing process for the dental appliance, the more weight can be awarded to the aggregated score for the lab, which is the lab quality score, and/or one or more of the sub-scores. In other words, the lab quality score for the dental lab can exceed some threshold score level if the dental appliance has a manufacturing difficulty level that exceeds some threshold level (especially if, for example, the dental appliance was generally shaped/sculpted accurately, the coloring was generally accurate, minimal corrections were made during a procedure to install the appliance, minimal types of corrections or easy corrections were made during the procedure, the patient was in the chair for less than a threshold period of time, etc.).

Additionally or alternatively, the computer system can weigh one or more of the sub-scores and/or the lab quality score based on a complexity of the dental appliance (block 224). Similar to what is described in block 222, the more complex the dental appliance, the more weight can be awarded to the aggregated score and/or one or more of the sub-scores. As another example, the less complex the dental appliance and the more adjustments made and/or inaccuracies identified during the manufacturing process, the lower the aggregated score and/or one or more sub-scores.

Additionally or alternatively, the computer system can weigh one or more of the sub-scores and/or the lab quality score based on a difficulty assessment for installing the dental appliance in patient mouths (block 226). Similar to what is described in block 222, the more complex or difficult to install the dental appliance, the more weight can be awarded to the aggregated score and/or one or more of the sub-scores, especially if minimal adjustments are made to the dental appliance during the procedure and/or the dental appliance is generally manufactured accurately/as expected.

Finally, the computer system can return the lab quality score that was determined for the respective dental lab in block 228. The score can be returned by storing it in the data store described herein. The computer system can transmit the score to one or more computing devices and/or systems, such as third party systems that may use the lab quality score to generate and provide information to relevant users. As an illustrative example, an insurance enterprise computing system can receive the lab quality score and use the score to determine reimbursement rates. As another example, the lab quality score can be provided to a data repository and computing system that provides customers, such as patients and dentists, with information about various labs. This information can be used by the customers to determine whether they would like to work with the dental lab to manufacture their dental appliance(s). Refer to FIGS. 5A-B for further discussion about exemplary graphical user interface (GUI) displays that may be provided to computing devices of various relevant users.

FIG. 3 is a flowchart of a process 300 for ranking a plurality of dental labs based on their respective quality scores and identifying one or more issues that may be addressed to improve quality scores and overall ranking for one or more of the plurality of dental labs. The process 300 can be performed by the remote computer system 122 described herein. In some implementations, the process 300 can be performed by other computing systems, devices, computers, networks, cloud-based systems, and/or cloud-based services. For illustrative purposes, the process 300 is described from the perspective of a computer system.

Referring to the process 300 in FIG. 3, the computer system can retrieve lab quality scores for a plurality of labs (block 302). The lab quality scores can be stored in a data store as described herein. The computer system can retrieve most recent or newest lab quality scores that are generated. The computer system can retrieve lab quality scores for a particular group of dental labs. For example, the computer system can retrieve lab quality scores for dental labs that are located in a particular geographic region (e.g., a geographic region that is closest to a particular dental office that is looking to work with a dental lab to manufacture their dental appliances). As another example, the computer system can retrieve lab quality scores for dental labs that manufacture a particular type of dental appliance (e.g., a type of dental appliance that a particular dental office is looking to have manufactured by a dental lab). The computer system can use one or more other criteria for selecting the lab quality scores to be retrieved in block 302.

The computer system can rank the plurality of labs into a list based on the respective lab quality scores satisfying one or more ranking criteria (block 304). The dental labs can be ranked from highest to lowest lab quality score. As another example, the dental labs' quality scores can be weighted and then ranked based on the weighted scores. The scores can be weighted based on or using a difficulty assessment for manufacturing the dental appliance. For example, the more difficulty or complex to manufacture the dental appliance, the more weight can be given to a particular lab quality score, especially if one or more sub-scores for the respective dental lab indicate that the lab is manufacturing dental appliances with quality (e.g., one or more of the sub-scores exceed one or more threshold score values). As another example, the less difficult or complex to manufacture the dental appliance, the less weight can be given to the particular lab quality score, especially if the one or more sub-scores for the respective dental lab are less than threshold score values.

In some implementations, the list of ranked labs can further be updated and/or re-ranked based on one or more sub-scores. For example, the list of ranked labs can be re-ranked or otherwise adjusted based on a combination of the lab quality scores and a score that corresponds to a total chair time of patients in dental offices who receive dental appliances manufactured by the respective dental labs. The less time patients spend in the chair, the higher the respective dental lab(s) can be ranked in the list, even if the dental lab(s) having the best sub-score for chair time (which means lowest total chair time) has a lower overall lab quality score than another dental lab. One or more other sub-scores described herein can also be used to rank or re-rank the dental labs in the list.

Sometimes, the one or more sub-scores can be used to filter out one or more dental labs from the list of ranked labs. For example, any dental lab that has a sub-score corresponding to quantity of dentist modifications that is less than a threshold score value can be removed from the list of ranked labs. In some implementations, a relevant user, such as a dentist, patient, or third party system can identify one or more sub-scores to be used in ranking and/or filtering the dental labs in the list of ranked labs.

The computer system can identify a lab in the list of ranked labs that has a respective quality score that is less than a threshold score value in block 306. For example, the computer system can identify a dental lab that is manufacturing dental appliances poorly and thus can be improved. The computer system can identify the lab in block 306 based on comparing the lab's overall quality score to the threshold score value. In some implementations, the computer system can identify the lab in block 306 based on comparing one or more sub-scores for the lab with one or more threshold score values. As described herein, the computer system can identify one or more suggestions or recommendations by which the lab identified in block 306 can improve their manufacturing practices. By improving one or more practices of the dental lab, the dental lab's overall quality score can increase. Increasing the dental lab's quality score can also cause the dental lab to increase in ranking in the list of ranked labs, which can result in making the dental lab more desirable to relevant users in the industry (e.g., dentists in dental offices, insurance companies, other third party systems, patients, etc.).

In block 308, the computer system can retrieve lab performance data for the identified lab. The data can be retrieved from a data store as described herein. The computer system can retrieve any other type of data about the dental lab, which can be generated by one or more lab technicians working in the dental lab and/or devices or machines in the dental lab. The devices and/or machines can be used for manufacturing dental appliances and can be configured to generate reports about a manufacturing process, including but not limited to an amount of time to manufacture a particular dental appliance, amount of modifications made to the manufacturing process, whether human intervention was needed, deviations from expected manufacturing instructions that were made during the actual manufacturing process, etc. The computer system can also retrieve any of the aggregated data described herein (e.g., refer to FIG. 1) and/or historic data about the identified dental lab. Sometimes, the computer system can also retrieve data about other labs, such as dental labs in a network of labs that includes the identified dental lab. The data about the other labs can be used to determine one or more suggestions or recommendations for improving performance of the identified lab.

The computer system can identify at least one issue in the identified lab based on the respective lab quality score and the lab performance data (or other aggregated data) (block 310). For example, the computer system can apply one or more rules and/or machine learning algorithms to the retrieved data to identify patterns in the data indicative of issue(s) in manufacturing performance of the dental lab. A machine learning model can be trained, for example, to identify patterns in the data that indicate the dental lab is taking too much time to manufacture a simple dental appliance. Another machine learning model can be trained to identify patterns in the data that indicate the dental lab is not finetuning their equipment, machines, or devices properly before manufacturing a type of dental appliance (which may result in more modifications being made by the dentist before, during, or after a procedure to insert the dental appliance into a patient's mouth). In yet some implementations, the retrieved data can include reports generated by relevant users that indicate one or more issues in the identified lab.

The computer system can generate, based on at least the identified issue(s), at least one recommendation to improve the identified lab in block 312. The computer system can apply one or more rules and/or machine learning algorithms to generate the at least one recommendation. A machine learning model can be trained, for example, to determine a set of possible ways in which a particular type of issue can be addressed or resolved at the identified lab. The machine learning model can further be trained to select one of the possible ways from the set that is most feasible for implementation at the identified lab (which can be based on type and/or amount of resources available at the identified lab, difficulty level of implementing the selected way to improve the dental lab, etc.) and/or likely to increase the quality score for the identified lab by at least a threshold amount. In some implementations, the computer system can analyze the data about other dental labs to determine what improvements or modifications were made to those labs in order to improve their respective quality scores. The computer system can perform one or more feasibility assessments using machine learning models to determine whether the improvements or modifications made to the other labs would have a similar or same effect in improving the quality score for the identified lab.

The computer system can generate and return output for the identified lab in block 314. The output can be provided to a user device of a relevant user, such as a manager or technician at the identified lab. The output can be presented in a graphical user interface (GUI) display at the user device. The output can include, for example, the lab quality score for the identified lab. The output can include the identified at least one issue for the lab. The output can include the at least one recommendation for improving the lab based on the lab quality score and/or the at least one issue. The output can include information about one or more other labs (e.g., labs in a relevant network of labs that includes the identified lab), their respective quality scores, and/or one or more ways in which they improved their procedures/practices, and, consequently, their quality scores. One or more other information can also be presented in the output. Refer to FIG. 5A for further discussion about outputting information about the identified lab.

The computer system can determine whether one or more other labs in the list have respective lab quality scores that are less than the threshold score value (block 316). If other labs have such low lab quality scores, the computer system can proceed to block 306 and repeat blocks 306-316 for each dental lab having a respective low quality score. If no other labs have such low quality scores, the computer system proceeds to block 318, in which the computer system can generate and return output indicating at least a subset of the labs in the list. The output generated and returned in block 318 can be similar to the output described in reference to FIG. 5A.

In some implementations, one or more blocks in the process 300 may be performed in response to receiving user input at the user device that presents the output. For example, the computer system can perform blocks 302-304 and 318. The user at the user device can review the output presented in response to block 318 and then select a particular lab in the list of ranked labs. Selection of the particular lab can cause the computer system to perform one or more of the blocks 308-314. Therefore, the computer system can efficiently use available compute resources to identify issues and recommendations for addressing those issues in a particular lab of interest to the user.

FIG. 4 is a flowchart of a process 400 for determining a dentist quality score. The process 400 can be performed by the remote computer system 122 described herein. In some implementations, the process 400 can be performed by other computing systems, devices, computers, networks, cloud-based systems, and/or cloud-based services. For illustrative purposes, the process 400 is described from the perspective of a computer system.

Referring to the process 400 in FIG. 4, the computer system can retrieve at least one lab quality score, dental appliance data, dentist procedure data for a dental appliance, and/or aggregated data in block 402. The computer system can determine what data to retrieve based on the dentist that is being scored. For example, the computer system can receive user input from a user device of a relevant user, such as a patient seeking to find a dentist to perform their dental procedure. The user input can include an indication of the patient's desire to view a quality score for a particular dentist or multiple dentists (e.g., dentists within a user-desired geographic range or region, dentists who perform a particular type of dental procedure that the user needs, etc.). The computer system can then retrieve relevant data from a data store described herein for the particular dentist or the multiple dentists identified by the user input.

As another example, the computer system can retrieve data for one or more dentists that satisfy one or more scoring criteria. The computer system can retrieve data for dentists that have not been scored within a threshold period of time. The computer system can retrieve data for dentists that have performed a threshold amount of dental procedures over a threshold period of time. The computer system can retrieve data for dentists that work with at least one dental lab that was recently scored or otherwise scored within a threshold period of time. Various other criteria can also be used to determine which dentists to score.

In block 404, the computer system can determine a dentist quality score based on the retrieved data. The dentist quality score can be determined using one or more rules and/or machine learning algorithms. The dentist quality score for a particular dentist can be determined based on aggregating, averaging, or summing one or more scores that were previously determined for one or more dental labs that are associated with or otherwise have a relationship with the dentist. Such associations and/or relationships can be defined in the aggregated data. The dentist quality score for the particular dentist can also be determined based on identifying relationships between one or more of the retrieved data and metrics that correspond to qualities of dentistry practice. The relationships can be identified and assessed using one or more rules and/or machine learning algorithms. As part of block 404, the computer system can perform one or more of blocks 406-414.

For example, in block 406, the computer system can determine whether a patient chair time associated with the dentist satisfies one or more chair time criteria. The computer system can retrieve dental office data associated with the dentist. The data can indicate a quantity of patients that go to the dentist for their dental appliance procedures. The data can indicate how long each of those patients remain in the chair during their respective dental appliance procedure, which indicates how long it takes the dentist to perform the procedure. Performing the procedure can include, for example, measuring and fitting the dental appliance into the patient's mouth, adjusting the dental appliance, and inserting the dental appliance. The data can also indicate an average chair time for patients of the dentist over some threshold period of time. Longer chair times can indicate that the dentist is performing more steps as part of the dental appliance procedure. The dentist may be inserting a complex dental appliance into the patient's mouth, thereby taking more time and precision/care. The dentist might have received a dental appliance from a dental lab that is inaccurate or not precise and thus the dentist may spend more time adjusting the dental appliance before inserting it into the patient's mouth. The computer system can analyze the total chair time data with other data, such as complexity of the dental appliance, accuracy of the dental lab in manufacturing the dental appliance, whether and how many patients return for adjustments, etc., to determine whether the total chair time satisfies the one or more chair time criteria.

If the criteria is satisfied, the computer system can determine a dentist quality score that exceeds some threshold score value. If the criteria is not satisfied, the computer system can determine a dentist quality score that is less than the threshold score value. For example, the more total chair time but the more complex the dental appliance and the lower frequency of returning patients for post-procedure adjustments, the higher the determined dentist quality score. This means that the dentist is a skilled, experienced, and/or of higher quality dentist compared to dentists having quality scores that are less than some threshold score value. As another example, the more total chair time but the less complex the dental appliance or the higher frequency of returning patients, the lower the determined dentist quality score. This means that the dentist is less skilled, experienced, and/or of lower quality compared to dentists having higher quality scores. As yet another illustrative example, the dentist can see more than a threshold quantity of patients but have an average chair time that is less than a threshold chair time and thus be assigned a dentist quality score that is greater than a threshold quality score value.

As another example, in block 408, the computer system can determine whether modifications to the dental appliance made by the dentist satisfy one or more modifications criteria. The computer system can retrieve dental procedure data. The data can indicate what types of modifications the dentist performs for one or more types of dental appliance procedures. The data can indicate how many modifications the dentist performs for the one or more types of procedures. The data can indicate complexity of the modifications that the dentist performs. The computer system can assess and aggregate the data to determine whether the modifications indicate higher quality or lower quality dentistry practices. For example, the more modifications performed on average, the lower the quality score for the dentist. As another example, the more modifications performed on average but the higher frequency that the dentist receives dental appliances that are not accurately manufactured, the higher the quality score for the dentist. As another example, the more complex the types of modifications performed, the higher the quality score for the dentist. As another example, the more complex the types of modifications performed for a complex dental appliance, and the lower frequency of returning patients post-procedure, the higher the quality score for the dentist. One or more other criteria may also be performed to determine whether the modifications performed by the dentist warrant a higher or lower quality score for the dentist.

As another example, in block 410, the computer system can determine whether one or more patients return to the dentist after a dental appliance procedure performed by the dentist. The computer system can retrieve dental office data, which can indicate how many patients return to the dental office over a threshold period of time, the reasons for their returns, and/or total chair time that each of the patients spend during their respective return visits. The computer system can assess and aggregate the retrieved data using the disclosed techniques to determine whether the patient returns to the dentist result in a higher or lower quality score for the dentist. For example, the higher frequency of returning patients, the lower quality score for the dentist. As another example, the higher frequency of returning patients for an issue with the dental appliance that stems from manufacturing the dental appliance rather than the dentist's actions during the procedure can result in a higher quality score for the dentist. As another example, regardless of the frequency of returning patients, an average chair time that is less than some threshold time can result in a higher quality score for the dentist. One or more other criteria can be used to determine the dentist quality score based on patient returns to the dentist after the dental appliance procedures performed by the dentist.

As yet another example, in block 412, the computer system can determine whether the lab quality score(s) associated with the dental appliance procedure(s) of the dentist satisfy one or more lab quality score criteria. The computer system can retrieve lab quality scores for one or more labs that work with the dentist being scored. The computer system can average the retrieved lab quality scores to determine the dentist quality score. The averaged lab quality score can be the dentist quality score. In some implementations, the computer system can select a lab quality score for a lab that works the most (e.g., highest frequency) with the dentist. If the selected lab quality score exceeds some threshold score value, then the computer system can assign a dentist quality score that is equal to the selected lab quality score or another score value that exceeds some threshold dentist quality score value. In some implementations, the computer system can select a highest lab quality score for a lab that works with the dentist and assign that score as the dentist quality score.

As another example, the computer system can weigh, in block 414, the dentist quality score based at least on a difficulty and/or complexity assessment of the dental appliance and/or the dental appliance procedure(s), as described above. Sometimes, the computer system can determine multiple sub-scores based on the determinations in blocks 406-412. The computer system can then average the sub-scores to determine the dentist quality score. As another example, the computer system can weight the sub-scores and then aggregate or summate the weighted sub-scores to determine the dentist quality score. The sub-scores can be weighted based on one or more factors. The sub-scores can also be weighted using one or more filters or preferences set by the relevant user who desires to score the dentist or view a quality score for the dentist.

In block 416, the computer system can return the dentist quality score for the dentist. The score can be stored in the data store in association with the dentist and/or one or more information about the dentist. The score can be provided to a user device to be outputted in a GUI display at the user device. The score can be used to rank the dentist amongst a set of dentists. The ranked dentists and their corresponding scores can then be outputted in the GUI display at the user device. Accordingly, in some implementations, the process 300 described in FIG. 3 can also be performed for ranking dentists based on their dentist quality scores and/or determining recommendations for the dentists to improve performance of their dental procedures and other dentistry practices.

FIGS. 5A-B are example graphical user interface (GUI) displays for outputting information about dental labs and/or dentists according to their respective quality scores. The GUIs described herein can be presented at user devices of relevant users, including but not limited to dental lab technicians, dental lab managers, dentists, patients, insurance providers, or other relevant third parties. The GUIs in FIGS. 5A-B are intended to be merely illustrative example GUIs. One or more information that is described in reference to the GUIs of FIGS. 5A-B can be presented in separate GUIs, in some implementations. The GUIs of FIGS. 5A-B can also present other information and/or display the information described herein in different ways and using different graphical elements, data fields, and other visual displays.

FIG. 5A illustrates example GUI 500 for presenting information about a set of dental labs and/or a set of dentists. The GUI 500 can output lab quality rankings 502, in which a set of dental labs can be ranked and presented based on their respective lab quality score. Additionally or alternatively, the GUI 500 can include dentist quality rankings 514, in which a set of dentists are ranked and presented based on their respective dentist quality score. In some implementations, the lab quality rankings 502 and the dentist quality rankings 514 can be presented in a separate GUIs.

A relevant user can sort the labs in the lab quality rankings table 502 using a selectable “sort labs” option 504 (e.g., button, control). Similarly, the user can sort the dentists in the dentist quality rankings table 514 by using a selectable “sort dentists” option 516 (e.g., button, control). As merely illustrative examples, the user can select the options 504 and/or 516 to sort the labs or dentists, respectively, based on dental appliance type and/or score value (e.g., from highest to lowest quality score, from lowest to highest quality score, etc.).

The relevant user can filter the labs in the lab quality rankings table 502 using a selectable “filter labs” option 506 (e.g., button, control). Similarly, the user can filter the dentists in the dentist quality rankings table 514 using the selectable “filter dentists” option 518 (e.g., button, control). As merely illustrative examples, the user can select the options 506 and/or 518 to filter the labs or dentists, respectively, based on a variety of factors, such as type of dental appliance, physical or geographic proximity to a location of the user, a threshold score value, etc.

The relevant user can select a “view lab information” option 508 in order to view additional information about one or more labs presented in the lab quality rankings table 502. Selecting the option 508 can cause the lab information to be presented in a pop-out window that visually overlays a portion of the GUI 500. Selecting the option 508 can cause the lab information to be presented in another, separate GUI. Selecting the option 508 can cause the lab information to be presented in the GUI 500.

The relevant user can select a “view dentist information” option 520 in order to view additional information about one or more dentists presented in the dentist quality rankings table 514. Selecting the option 520 can cause the dentist information to be presented in a pop-out window that visually overlays a portion of the GUI 500. Selecting the option 520 can cause the dentist information to be presented in another, separate GUI. Selecting the option 520 can cause the dentist information to be presented in the GUI 500.

The GUI 500 may also include a data field 512 for the relevant user to input their location or desired location. Once the user provides their location or geographic region of interest, the GUI 500 can be automatically updated and populated with information relevant to the user's geographic location. For example, the lab quality rankings table 502 can be updated to present only labs that are within a threshold distance or geographic range from the user's inputted location information. As another example, the dentist quality rankings 514 can be updated to present only dentists within the threshold distance or geographic range from the user's inputted location information.

The GUI 500 may include a labs map view 510, which can visually display one or more of the labs listed in the table 502 relative to the location of the user (or the user inputted location information in the data field 512). The GUI 500 may include a dentist map view 522, which can visually display one or more of the dentists listed in the table 514 relative to the location of the user (or the user inputted location information in the data field 512). In some implementations, the user can select one or more of the labs or dentists presented in the respective maps 510 and 522 to view additional information about the selected lab or dentist. The additional information can be presented in a pop-out window visually overlaying a portion of the GUI 500. The additional information can also be presented in a separate GUI.

FIG. 5B illustrates example GUI 550 for presenting information about a specific dental lab. Similar GUIs can also be used to present information about a specific dental office. The GUI 550 can output a quality score 552 for the particular dental lab. The GUI 550 can also output selectable options 554, 556, 558, 562, and/or 564 (e.g., buttons, controls). Selecting any one of the options 554, 556, 558, 562, and 564 can cause corresponding information to be presented in the GUI 550, as a pop-out window visually overlaying a portion of the GUI 500, and/or in a new GUI.

Selecting the option 554 can cause information to be presented about the particular lab's ranking amongst a set of labs. The particular lab can be ranked amongst a set of labs that are in a same geographic region and/or a set of labs that manufacture a same type of dental appliance. The particular lab can be ranked amongst other sets of labs based on one or more filtering criteria as described throughout this disclosure.

Selecting the option 556 can cause information to be presented about a breakdown of the particular lab's quality score. For example, the information can include one or more sub-scores that were used to generate the particular lab's quality score. The information can include further analysis and/or description about how the particular lab's quality score was calculated and/or what data was used to determine the quality score.

Selecting the option 558 can cause information to be presented about one or more of the sub-scores that were used to determine the particular lab's quality score. For example, the information can include a description of each sub-score, a value of each sub-score, and/or a weighting of each sub-score. The information can also include details about how each sub-score was determined and/or how each sub-score impacts the overall quality score for the dental lab.

Selecting the option 562 can cause one or more recommendations to be presented that can be implemented by the particular lab to improve their practice and/or performance. The recommendations can include descriptions about one or more issues that were identified in the particular lab and/or how one or more recommended actions can be taken to improve the practice and performance of the lab, and consequently improve the lab's quality score.

Selecting the option 564 can cause at least one of the recommendations to be implemented in order to improve the particular lab's practice/performance. The at least one recommendation can be automatically implemented by any of the relevant computer systems described herein. As another example, selecting the option 564 can cause any of the relevant computer systems to generate instructions that are presented to the relevant user for implementing the at least one recommendation. The relevant user can then implement the recommendation(s) accordingly.

The GUI 550 can also present an identified areas of improvement table 560. The table 560 can indicate one or more issues that have been identified for the particular lab using the disclosed techniques. The table 560 can list a threshold quantity of identified issues, such as a top 5 issues. As another example, the table 560 can present a list of issues that, if fixed, would have the greatest impact on the lab's quality score and/or cause at least a threshold increase in the lab's quality score if fixed. As yet another example, the table 560 can present a list of issues that are most important to be fixed, which can be determined based on one or more criteria.

The illustrative table 560 lists at least 4 issues that have been identified for the particular lab. The table 560 presents, for each issue, a type of improvement area, a description of the issue, and a potential score impact. The table 560 can include one or more other information described throughout this disclosure. For example, the table 560 can include one or more sub-scores that were used in assessing and identifying the improvement areas. The issues in the table 560 are sorted based on impact that each issue has on the lab's quality score. As a result, an issue that would have the greatest impact on the quality score (e.g., would increase the quality score by the greatest amount if fixed) is presented first in the table 560 and an issue that would have the smallest impact on the quality score can be presented last in the table 560. The issues in the table 560 can also be sorted based on one or more other criteria, including but not limited to user preferences (which can be provided as user input at the GUI 550).

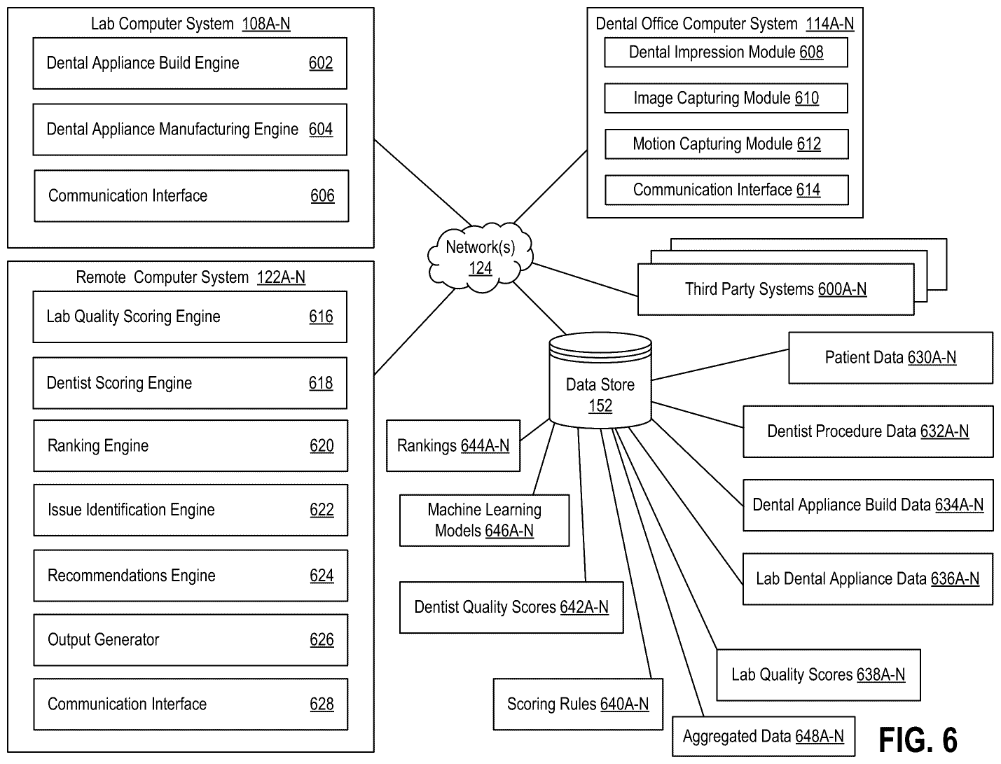

FIG. 6 is a system diagram of one or more components that can be used to perform the disclosed techniques. The lab computer systems 108A-N, remote computer systems 122A-N, dental office computer systems 114A-N, and data store 152 can communicate (e.g., wirelessly, wired) with each other via the network(s) 124, as described herein. Additionally or alternatively, any of these components can also communicate with one or more third party systems 600A-N via the network(s) 124. One or more of these components can be part of a same computing system, computing device, and/or network of devices or systems. As illustrated in FIG. 6, one or more of these components can also be separate systems and/or devices in communication with each other.

Each lab computer system 108A-N can include a dental appliance build engine 602, a dental appliance manufacturing engine 604, and a communication interface 606. These components of the lab computer system 108 are merely illustrative. The lab computer system 108 can include one or more additional, fewer, or other components to perform the disclosed techniques.

The dental appliance build engine 602 can be configured to generate build instructions for dental appliances to be manufactured at the dental lab that corresponds to the lab computer system 108. In some implementations, the build instructions can be generated by the dental office computer systems 114A-N and/or the remote computer systems 122A-N. The engine 602 can then receive the build instructions from either computer system. In some implementations, the build instructions can be generated by any of the computer systems described herein then stored as dental appliance build data 634A-N in the data store 152. The dental appliance build engine 602 can then retrieve the data 634A-N and determine, generate, and/or update/modify the build instructions before the dental appliance is manufactured.

The dental appliance manufacturing engine 604 can receive the dental appliance build data 634A-N from the engine 602 and/or the build instructions from the engine 602. The engine 604 can be configured to manufacture the dental appliance according to the build instructions. For example, the engine 604 can control one or more manufacturing devices, components, and/or machines (e.g., 3D printers, fabrication machines, etc.) in the respective dental lab to execute the build instructions and thus build the dental appliance. In some implementations, the engine 604 can translate the build instructions into machine-readable code or instructions to be executed by each of the manufacturing devices, components, and/or machines that are used for manufacturing/building the particular dental appliance.

The dental appliance manufacturing engine 604 can generate lab dental appliance data 636A-N(which can be stored in the data store 152) before, during, and after manufacturing the dental appliance. For example, the data 636A-N can describe the manufacturing process for the particular dental appliance, including but not limited to what materials were used to manufacture the dental appliance, how long it took to manufacture the dental appliance, whether human intervention was needed during manufacturing, what type of human intervention or modifications were made during manufacturing, what human modifications were made to the dental appliance post-manufacturing, a difficulty level of manufacturing the dental appliance, a type of dental appliance that was manufactured, etc. In some implementations, one or more of the data 636A-N can include user input that is provided by a lab technician or other relevant user at the dental lab and to the respective lab computer system 108. The user input can indicate any one or more of the information mentioned above.

The communication interface 606 can provide communication between the components of the lab computer system 108 and/or one or more other systems and/or devices described herein.

Each dental office computer system 114A-N can include a dental impression module 608, an image capturing module 610, a motion capturing module 612, and a communication interface 614. These components of the dental office computer system 114 are merely illustrative. The dental office computer system 114 can include one or more additional, fewer, or other components to perform the disclosed techniques.

The dental impression module 608 can be configured to control the dental impression station 116 described in FIG. 1. The module 608 can generate data based on impressions that are taken using the dental impression station 116.

The image capturing module 610 can be configured to control the image capture system 118 described in FIG. 1. The module 610 can generate data, such as image and/or video data of a patient's teeth based on the images captured by the system 118.

The motion capturing module 612 can be configured to control the motion capture system 120 described in FIG. 1. The module 612 can generate data, such as motion data and/or a 3D model of the patient's teeth based on the motion captured and recorded by the system 120.

The communication interface 614 can provide communication between the components of the dental office computer system 114 and/or one or more other systems and/or devices described herein.

Any of the data that is generated by the dental office computer system 114 can be stored in the data store 152 as patient data 630A-N, dentist procedure data 632A-N, and/or the dental appliance build data 634A-N. For example, the patient data 630A-N can include one or more of the dental impressions, images, motions, and/or 3D models of the particular patient's teeth/mouth. The patient data 630A-N can also include historic data about the patient's prior dental conditions, prior dental office visits, prior dental appliances, prior dental appliance procedures, etc. Any of the patient data 630A-N can be automatically generated by components of the dental office computer system 114 and/or provided as user input to the computer system 114 by a relevant user, such as a dentist or other relevant user.

The dental appliance build data 634A-N can be generated by any one or more components of the computer system 114. For example, the computer system 114 can generate a 3D model of the patient's mouth using the data from the modules 608-612. Using the 3D model and the respective patient data 630A-N, the computer system 114 can design a dental appliance for the patient. Designing the dental appliance may also include generating build instructions, which can then be used by the lab computer system 108 to manufacture the dental appliance for the patient.

The dentist procedure data 632A-N can be generated by any one or more components of the computer system 114. One or more of the data 632A-N can additionally or alternatively be provided as user input by one or more relevant users in the dental office. The dentist procedure data 632A-N can indicate, for each patient, a type of dental procedure, a type of dental appliance being installed in the patient's mouth, steps that were performed when installing the dental appliance into the patient's mouth, a total chair time of the patient, whether the dentist performed any modifications to the dental appliance to fit the appliance into the patient's mouth, etc. Any one or more other data can also be generated as part of the dentist procedure data 632A-N described herein.

Each remote computer system 122A-N can include a lab quality scoring engine 616, a dentist scoring engine 618, a ranking engine 620, an issue identification engine 622, a recommendations engine 624, an output generator 626, and a communication interface 628. These components of the remote computer system 122 are merely illustrative. The remote computer system 122 can include one or more additional, fewer, or other components to perform the disclosed techniques. In some implementations, the system described herein can include one remote computer system 122 that communicates with all or a subset of the lab computer systems 108A-N and/or the dental office computer systems 114A-N. In some implementations, each dental lab and/or dental office can be associated with a different remote computer system 122A-N. Various other implementations are also possible.

The lab quality scoring engine 616 can be configured to determine lab quality scores for the dental labs described throughout this disclosure. The engine 616 can retrieve or receive data from any of the computer systems described herein and/or the data store 152 for determining a lab quality score. For example, the engine 616 can retrieve scoring rules 640A-N, the lab dental appliance data 636A-N, the dental appliance build data 634A-N, the dentist procedure data 632A-N, and/or the patient data 630A-N. Any of this data can then be aggregated, as aggregated data 648A-N, as described herein to determine and generate lab quality scores for the respective dental labs. The scoring rules 640A-N can be used to determine how to aggregate, combine, or otherwise analyze the received or retrieved data in order to generate the lab quality scores. The engine 616 can also use one or more machine learning models 646A-N to aggregate the data and determine the lab quality scores for the dental labs. The determined scores can be stored in the data store 152 as lab quality scores 638A-N. Refer to the process 200 in FIGS. 2A-B for further discussion about how the engine 616 can determine the lab quality scores.

The dentist scoring engine 618 can be configured to determine dentist quality scores for dentists described throughout this disclosure. The engine 618 can retrieve or receive data from any of the computer systems described herein and/or the data store 152 for determining a dentist quality score. For example, the engine 618 can retrieve one or more of the scoring rules 640A-N, the lab dental appliance data 636A-N, the dental appliance build data 634A-N, the dentist procedure data 632A-N, the patient data 630A-N, and/or the lab quality scores 638A-N. Any of this data can then be aggregated, as aggregated data 648A-N, as described herein to determine and generate dentist quality scores for the respective dentist. The one or more scoring rules 640A-N can be used to determine how to aggregate, combine, or otherwise analyze the received or retrieved data in order to generate the dentist quality scores. The engine 618 can also use one or more machine learning models 646A-N to aggregate the data and determine the dentist quality scores. The determined scores can be stored in the data store 152 as dentist quality scores 642A-N. Refer to the process 400 in FIG. 4 for further discussion about determining dentist quality scores.

The ranking engine 620 can be configured to rank one or more dental labs and/or one or more dentists into lists. The ranking engine 620 can rank the dental labs and/or dentists based on their respective quality scores, geographic regions, or other factors described herein. The engine 620 can retrieve, from the data store 152, any one or more of the dentist quality rules 642A-N, the lab quality scores 638A-N, and/or other information about the dentists and/or dental labs for purposes of ranking them. The engine 620 can also receive any of this data from computer systems and/or engines described herein, such as the lab quality scoring engine 616 and/or the dentist scoring engine 618. The engine 620 can generate a list of ranked dental labs and/or a list of ranked dentists and store the list(s) as rankings 644A-N in the data store 152. Refer to the process 300 in FIG. 3 for further discussion about ranking the dental labs. As described herein, the process 300 can also be used to rank the dentists.

The issue identification engine 622 can be configured to identify one or more issues with a particular dental lab and/or dentist that caused the dental lab and/or dentist to have a low quality score. The engine 622 can retrieve information/data from the data store 152, such as the lab quality scores 638A-N, the dentist quality scores 642A-N, the lab rankings 644A-N, the dentist procedure data 632A-N, and/or the dental appliance data 636A-N. The engine 622 can then apply one or more machine learning models 646A-N to analyze the retrieved data and identify one or more issues or areas for improvement. In some implementations, the issues identified by the engine 622 can be stored in the data store 152 in association with the particular dental lab and/or dentist (not depicted). Refer to the process 300 in FIG. 3 for further discussion about identifying issues.

The recommendations engine 624 can receive the identified issues from the issue identification engine 622 and can be configured to generate one or more recommendations about how the dental lab and/or the dentist can improve or otherwise resolve the identified issues. The engine 624 can apply one or more machine learning models 646A-N to generate the recommendations. The engine 624 can also retrieve from the data store 152 historic data (not depicted) associated with the particular dental lab, dentist, or other labs or dentists that experienced similar or same issues. This data may also be used by the engine 624 to determine the recommendations. The engine 624 can also generate information such as an effect that implementing the recommendations would have on the lab's respective quality score and/or the dentist's respective quality score. In some implementations, the recommendations generated by the engine 624 can be stored in the data store 152 in association with the particular dental lab and/or dentist (not depicted). Refer to the process 300 in FIG. 3 for further discussion about determining recommendations to improve issues identified for the dental lab and/or dentist.

The output generator 626 can be configured to generate information that can be presented at devices of relevant users. For example, the generator 626 can generate instructions that cause user computing devices of patients, third parties, dentists, and/or dental labs to present output in respective graphical user interface (GUI) displays that include one or more lab quality scores, dentist quality scores, rankings, dental appliance data, dentist procedure data, and/or patient data. The generator 626 can also receive the identified issues and recommendations from the issue identification engine 622 and the recommendations engine 624, respectively, and then present such information in the output. Refer to FIGS. 5A-B for illustrative GUI output that can be generated and provided to relevant users described herein.

The communication interface 628 can provide communication between the components of the remote computer system 122 and/or one or more other systems and/or devices described herein.

The third party systems 600A-N can correspond to any type of third party that may utilize the information determined and/or provided by the disclosed techniques. For example, the third party systems 600A-N can include computing systems of insurance providers and insurance companies. As an illustrative example, the insurance providers and/or companies may use the lab quality scores 638A-N and/or the lab rankings 644A-N to determine that labs with particular scores or rankings are paid higher amounts because in the long run, they save money since those labs manufacture higher quality dental appliances. Such a determination may also drive future performance improvements amongst all of the dental labs. The third party systems 600A-N can correspond to one or more other types of third parties, including but not limited to reimbursors, and systems providing/connecting opportunities across patients, dentists, and/or labs.

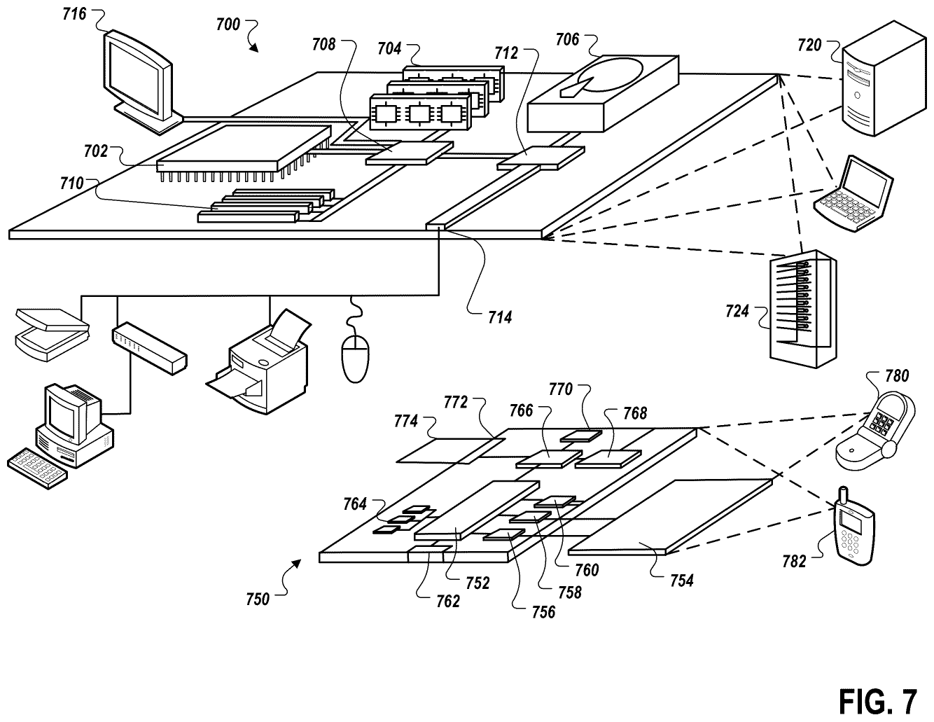

FIG. 7 shows an example of a computing device 700 and an example of a mobile computing device that can be used to implement the techniques described here. The computing device 700 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 700 includes a processor 702, a memory 704, a storage device 706, a high-speed interface 708 connecting to the memory 704 and multiple high-speed expansion ports 710, and a low-speed interface 712 connecting to a low-speed expansion port 714 and the storage device 706. Each of the processor 702, the memory 704, the storage device 706, the high-speed interface 708, the high-speed expansion ports 710, and the low-speed interface 712, are interconnected using various busses, and can be mounted on a common motherboard or in other manners as appropriate. The processor 702 can process instructions for execution within the computing device 700, including instructions stored in the memory 704 or on the storage device 706 to display graphical information for a GUI on an external input/output device, such as a display 716 coupled to the high-speed interface 708. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 704 stores information within the computing device 700. In some implementations, the memory 704 is a volatile memory unit or units. In some implementations, the memory 704 is a non-volatile memory unit or units. The memory 704 can also be another form of computer-readable medium, such as a magnetic or optical disk.

The storage device 706 is capable of providing mass storage for the computing device 700. In some implementations, the storage device 706 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 704, the storage device 706, or memory on the processor 702.

The high-speed interface 708 manages bandwidth-intensive operations for the computing device 700, while the low-speed interface 712 manages lower bandwidth-intensive operations. Such allocation of functions is exemplary only. In some implementations, the high-speed interface 708 is coupled to the memory 704, the display 716 (e.g., through a graphics processor or accelerator), and to the high-speed expansion ports 710, which can accept various expansion cards (not shown). In the implementation, the low-speed interface 712 is coupled to the storage device 706 and the low-speed expansion port 714. The low-speed expansion port 714, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 700 can be implemented in a number of different forms, as shown in the figure. For example, it can be implemented as a standard server 720, or multiple times in a group of such servers. In addition, it can be implemented in a personal computer such as a laptop computer 722. It can also be implemented as part of a rack server system 724. Alternatively, components from the computing device 700 can be combined with other components in a mobile device (not shown), such as a mobile computing device 750. Each of such devices can contain one or more of the computing device 700 and the mobile computing device 750, and an entire system can be made up of multiple computing devices communicating with each other.

The mobile computing device 750 includes a processor 752, a memory 764, an input/output device such as a display 754, a communication interface 766, and a transceiver 768, among other components. The mobile computing device 750 can also be provided with a storage device, such as a micro-drive or other device, to provide additional storage. Each of the processor 752, the memory 764, the display 754, the communication interface 766, and the transceiver 768, are interconnected using various buses, and several of the components can be mounted on a common motherboard or in other manners as appropriate.

The processor 752 can execute instructions within the mobile computing device 750, including instructions stored in the memory 764. The processor 752 can be implemented as a chipset of chips that include separate and multiple analog and digital processors. The processor 752 can provide, for example, for coordination of the other components of the mobile computing device 750, such as control of user interfaces, applications run by the mobile computing device 750, and wireless communication by the mobile computing device 750.

The processor 752 can communicate with a user through a control interface 758 and a display interface 756 coupled to the display 754. The display 754 can be, for example, a TFT (Thin-Film-Transistor Liquid Crystal Display) display or an OLED (Organic Light Emitting Diode) display, or other appropriate display technology. The display interface 756 can comprise appropriate circuitry for driving the display 754 to present graphical and other information to a user. The control interface 758 can receive commands from a user and convert them for submission to the processor 752. In addition, an external interface 762 can provide communication with the processor 752, so as to enable near area communication of the mobile computing device 750 with other devices. The external interface 762 can provide, for example, for wired communication in some implementations, or for wireless communication in other implementations, and multiple interfaces can also be used.

The memory 764 stores information within the mobile computing device 750. The memory 764 can be implemented as one or more of a computer-readable medium or media, a volatile memory unit or units, or a non-volatile memory unit or units. An expansion memory 774 can also be provided and connected to the mobile computing device 750 through an expansion interface 772, which can include, for example, a SIMM (Single In Line Memory Module) card interface. The expansion memory 774 can provide extra storage space for the mobile computing device 750, or can also store applications or other information for the mobile computing device 750. Specifically, the expansion memory 774 can include instructions to carry out or supplement the processes described above, and can include secure information also. Thus, for example, the expansion memory 774 can be provide as a security module for the mobile computing device 750, and can be programmed with instructions that permit secure use of the mobile computing device 750. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 764, the expansion memory 774, or memory on the processor 752. In some implementations, the computer program product can be received in a propagated signal, for example, over the transceiver 768 or the external interface 762.

The mobile computing device 750 can communicate wirelessly through the communication interface 766, which can include digital signal processing circuitry where necessary. The communication interface 766 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 768 using a radio-frequency. In addition, short-range communication can occur, such as using a Bluetooth, WiFi, or other such transceiver (not shown). In addition, a GPS (Global Positioning System) receiver module 770 can provide additional navigation- and location-related wireless data to the mobile computing device 750, which can be used as appropriate by applications running on the mobile computing device 750.

The mobile computing device 750 can also communicate audibly using an audio codec 760, which can receive spoken information from a user and convert it to usable digital information. The audio codec 760 can likewise generate audible sound for a user, such as through a speaker, e.g., in a handset of the mobile computing device 750. Such sound can include sound from voice telephone calls, can include recorded sound (e.g., voice messages, music files, etc.) and can also include sound generated by applications operating on the mobile computing device 750.

The mobile computing device 750 can be implemented in a number of different forms, as shown in the figure. For example, it can be implemented as a cellular telephone 780. It can also be implemented as part of a smart-phone 782, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of the disclosed technology or of what may be claimed, but rather as descriptions of features that may be specific to particular embodiments of particular disclosed technologies. Certain features that are described in this specification in the context of separate embodiments can also be implemented in combination in a single embodiment in part or in whole. Conversely, various features that are described in the context of a single embodiment can also be implemented in multiple embodiments separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination. Similarly, while operations may be described in a particular order, this should not be understood as requiring that such operations be performed in the particular order or in sequential order, or that all operations be performed, to achieve desirable results. Particular embodiments of the subject matter have been described. Other embodiments are within the scope of the following claims.

What is claimed is:

1. A system for determining a lab quality score, the system comprising:
a dental lab computer system that is configured to:
generate instructions that, when executed, cause at least one manufacturing machine to manufacture a dental appliance for a patient in a dental lab; and
generate dental appliance data based at least in part on user input that is received from a lab technician indicating information about manufacturing the dental appliance for the patient;
a dental office computer system that is configured to generate dental appliance procedure data based on a dentist performing a procedure to install the dental appliance in the patient's mouth; and
a remote computer system in communication with the dental lab computer system and the dental office computer system, wherein the remote computer system is configured to:
receive the dental appliance data and the dental appliance procedure data from the dental lab computer system and the dental office computer system, respectively;
aggregate the received data;
determine a lab quality score for the dental lab based at least in part on the aggregated data;
identify, based on the lab quality score, at least one quality issue for the dental lab;
generate at least one recommendation to improve the lab quality score of the dental lab based on the identified at least one quality issue; and
return the lab quality score, the identified at least one quality issue, and the at least one recommendation for presentation in a graphical user interface (GUI) display of a computing device of a relevant user.

2. The system of claim 1, wherein determining the lab quality score for the dental lab comprises:
generating a set of sub-scores; and
aggregating the sub-scores to determine the lab quality score.

3. The system of claim 2, wherein generating the set of sub-scores comprises: generating a sub-score that exceeds a threshold sub-score value based on an accuracy in shaping or sculpting the dental appliance at the dental lab satisfying one or more manufacturing criteria.

4. The system of claim 2, wherein generating the set of sub-scores comprises: generating a sub-score that exceeds a threshold sub-score value based on an accuracy of coloring the dental appliance at the dental lab satisfying one or more color criteria.

5. The system of claim 2, wherein generating the set of sub-scores comprises: generating a sub-score that exceeds a threshold sub-score value based on manufacturing practices of the dental lab satisfying one or more manufacturing criteria.

6. The system of claim 2, wherein generating the set of sub-scores comprises: generating a sub-score that exceeds a threshold sub-score value based on a quantity of corrections made to the dental appliance by the dentist during the procedure to install the dental appliance in the patient's mouth being less than a threshold quantity of corrections.

7. The system of claim 2, wherein generating the set of sub-scores comprises: generating a sub-score that exceeds a threshold sub-score value based on a type of corrections made to the dental appliance by the dentist during the procedure to install the dental appliance in the patient's mouth satisfying one or more correction-type criteria.

8. The system of claim 2, wherein generating the set of sub-scores comprises: generating a sub-score that exceeds a threshold sub-score value based on a total chair time of the patient during the procedure to install the dental appliance in the patient's mouth being less than a threshold amount of time.

9. The system of claim 2, wherein aggregating the sub-scores to determine the lab quality score comprises: weighting one or more of the sub-scores based on a difficulty assessment for manufacturing the dental appliance, wherein the lab quality score exceeds a threshold score value when manufacturing the dental appliance has a difficulty level above a threshold difficulty level.

10. The system of claim 2, wherein aggregating the sub-scores to determine the lab quality score comprises: weighting one or more of the sub-scores based on a complexity of the dental appliance, wherein the lab quality score exceeds a threshold score value when the complexity of the dental appliance is greater than a threshold level of complexity.

11. The system of claim 1, wherein the remote computer system is further configured to determine a dentist quality score for the dentist based on at least one of the aggregated data or the lab quality score.

12. The system of claim 11, wherein determining the dentist quality score for the dentist comprises: assigning the dentist quality score a value that exceeds a threshold score value based on a total patient chair time associated with the dentist being less than a threshold chair time value.

13. The system of claim 11, wherein determining the dentist quality score for the dentist comprises: assigning the dentist quality score a value that exceeds a threshold score value based on the lab quality score exceeding a threshold lab quality score value.

14. The system of claim 1, wherein the remote computer system is further configured to:
retrieve, from a data store, lab quality scores for a plurality of dental labs that includes the dental lab; rank the plurality of dental labs that includes the dental lab based on the respective lab quality scores into a list of ranked dental labs; and
return the list of ranked dental labs.

15. The system of claim 1, wherein the relevant user is another patient.

16. The system of claim 1, wherein the relevant user is a third party insurance provider.

17. The system of claim 1, wherein the relevant user is another dentist or another dental lab.

18. A method for determining a lab quality score, the method comprising:
receiving, by a remote computer system, dental appliance data and dental appliance procedure data from a dental lab computer system and a dental office computer system, respectively, wherein the dental appliance data is based at least in part on user input that is received from a lab technician at the dental lab computer system indicating information about manufacturing a dental appliance for a patient, wherein the dental appliance procedure data is based on a dentist performing a procedure to install the dental appliance in the patient's mouth;
aggregating, by the remote computer system, the received data;
determining, by the remote computer system, a lab quality score for a dental lab that is associated with the dental appliance data based at least in part on the aggregated data;
identifying, by the remote computer system and based on the lab quality score, at least one quality issue for the dental lab;
generating, by the remote computer system, at least one recommendation to improve the lab quality score of the dental lab based on the identified at least one quality issue; and
returning, by the remote computer system, the lab quality score, the identified at least one quality issue, and the at least one recommendation for presentation in a graphical user interface (GUI) display of a computing device of a relevant user.
